(19) United States
(12) Patent Application Publication
(10) Pub. No.: US 2019/0225657 A1
(43) Pub. Date: Jul. 25, 2019

(54) METHOD FOR THE PRODUCTION OF HAPLOID AND SUBSEQUENT DOUBLED HAPLOID PLANTS

(71) Applicant: Keygene N.V., Wageningen (NL)

(72) Inventors: Rik Hubertus Martinus OP DEN CAMP, Wageningen (NL); Peter Johannes VAN DIJK, Wageningen (NL); Anthony GALLARD, Wageningen (NL)

(73) Assignee: Keygene N.V., Wageningen (NL)

(21) Appl. No.: 16/302,949

(22) PCT Filed: May 19, 2017
(86) PCT No.: PCT/NL2017/050320

§ 371 Date: Nov. 19, 2018

(30) Foreign Application Priority Data
May 20, 2016 (NL) 2016806

(51) Int. Cl. C07K 14/415; A01H 6/46; A01H 1/08

(52) U.S. Cl. CPC C07K 14/415; A01H 1/08; A01H 6/4636

(57) ABSTRACT

The present disclosure provides a modified CenH3 protein that, when present in a plant, allows the plant to be used as a haploid inducer line for plant breeding purposes. Polynucleotides encoding such modified CenH3 proteins, chimeric genes and vectors comprising such polynucleotides, host cells, and plants comprising such polynucleotides, chimeric genes or vectors are also provided. Additionally, methods for making such plants as well as methods for producing haploid or doubled haploid plants using such plants are disclosed.

### FIELD OF THE INVENTION

The disclosure relates to the field of agriculture. In particular, the disclosure relates to CenH3 proteins and polynucleotides encoding them, methods for the production of haploid as well as subsequent doubled haploid plants, and plants and seeds derived thereof.

### BACKGROUND OF THE INVENTION

A high degree of heterozygosity in breeding material can make plant breeding and selection for beneficial traits a very time consuming process. Extensive population screening, even with the latest molecular breeding tools, is both laborious and costly.

The creation of haploid plants followed by chemical or spontaneous genome doubling has proven to be an efficient way to solve the problem of high heterozygosity and accelerate the breeding process. Such technology is also referred to as ‘doubled haploid production system’. The use of the doubled haploid production system has allowed breeders to achieve homozygosity at all loci in a single generation via whole-genome duplication. This effectively obviates the need for selfing or backcrossing, where normally at least 7 generations of selfing or backcrossing would be needed to reduce the heterozygosity to an acceptable level.

Haploid plants can be generated according to different methodologies. For instance, haploid plants can be produced in some crops by using a method referred to as ‘microspore culture’.

However, this method is costly, time-consuming, and does not work in all crops. In some crop species, (doubled) haploid plants can be obtained by parthenogenesis of the egg cell or by elimination of one of the parental genomes. However, such methods are not optimal as they only work in few selected crop species and yield rather low rates of (doubled) haploid plants.

WO2011044132 discloses a method for producing haploid plants consisting of inactivating or altering or knocking out the centromere-specific H3 (CenH3) protein in a plant. In a first step, the method consists of eliminating or knocking down the endogenous CenH3 gene in plant. In a second step, an expression cassette encoding a mutated or altered CenH3 protein is introduced in the plant. The mutated or altered CenH3 protein is generated by fusing an, optionally GFP-tagged, H3.3 N-terminal domain to the endogenous CenH3 histone-fold domain. Such methodology is also known as ‘GFP-tailswap’ or ‘tailswap’ (also reviewed in Britt and Kuppu, Front Plant Sci. 2016; 7: 357). The crossing of the plant harbouring such (GFP-)tailswap with a wild type plant (i.e. having functional endogenous CenH3 protein without a (GFP-)tailswap), causes uniparental genome elimination, which in turn results in the production of a haploid plant. Some haploid induction, though less frequent, was also found with N-terminal addition of GFP to endogenous CenH3 (no “tailswap”). However, this methodology is not ideal as it laborious, time-consuming and requires to generate a transgenic plant. Furthermore, this method has only been demonstrated in the model plant Arabidopsis thaliana and not in crop plants.

WO2014110274 describes a method for producing haploid plants consisting of crossing a first plant expressing an endogenous CenH3 gene to a second plant referred to as a haploid inducer plant having a genome from at least two species, wherein a majority of the genome is from a first species and the genome comprises a heterologous genomic region from a second species, wherein the heterologous genomic region encodes a CenH3 polypeptide different from the CenH3 of the first species (also described in Maheshwari et al, PLoS Genet. 2015 Jan. 26; 11(1):e1004970)). However, this methodology is not optimal as it suffers from the same pitfall as above, i.e. laborious, time-consuming and requires to generate a transgenic plant. Further, the method is associated with low yield of haploid plants.

Other methods consist of introducing one or more point mutations leading to single amino acid change in the C-terminal histone fold domain of CenH3 protein or CenH3 gene coding the CenH3 protein. Examples of such mutations in the C-terminal histone fold domain of the CenH3 protein were reported in Karimi-Ashtiyani et al (2015) PNAS Vol: 112, pages 11211-11216; Kuppu, et al. PLOS Genetics (2015) http://dx.doi.org/10.1371/journal.pgen.1005494. However, the success of such methods is mitigated as some, as not all of these mutations were found to be sufficient to induce uniparental genome elimination after crossing with a wild type plant to produce a haploid plant.

Therefore, it remains elusive which mutation(s) or modification(s) in the CenH3 protein or CenH3 gene coding for the CenH3 protein are capable or sufficient to induce uniparental genome elimination to produce haploid plants. Thus, there remains a need in the art for alternative or improved methods that allow efficient generation of haploid plants (e.g. less labour-intensive, less-time consuming, less expensive, and/or do not necessarily require making a transgenic plant), which can subsequently be doubled to produce doubled haploid plants. With doubled haploid production systems, homozygosity may be achieved in one generation.

### SUMMARY OF THE INVENTION

In a first aspect, the present invention relates to a CenH3 protein of plant origin comprising one or more active mutations in its N-terminal tail domain.

In an embodiment, the one or more active mutations may be present in the CenH3 motif block 1.

In an embodiment, the one or more active mutations may be present:

a) in a protein comprising the amino acid sequence of SEQ ID NO: 1, or in a variant thereof having at least 70%, more preferably at least 80%, even more preferably at least 90%, yet even more preferably at least 95%, most preferably at least 97%, 98% or 99% sequence identity to the amino acid sequence of SEQ ID NO: 1; or

b) in a protein comprising the amino acid sequence of SEQ ID NO: 4; or

c) in a protein comprising the amino acid sequence of SEQ ID NO: 2, or in a variant thereof having at least 70%, more preferably at least 80%, even more preferably at least 90%, yet even more preferably at least 95%, most preferably at least 97%, 98% or 99% sequence identity to the amino acid sequence of SEQ ID NO: 2; or

d) in a protein comprising the amino acid sequence of SEQ ID NO: 5.

In an embodiment, the active mutation may be:

a) in the amino acid residue at position 10 of the amino acid sequence of SEQ ID NO: 1, or in a variant thereof having at least 70%, more preferably at least 80%, even more preferably at least 90%, yet even more preferably at least 95%, most preferably at least 97%, 98% or 99% sequence identity to the amino acid sequence of SEQ ID NO: 1; or

b) in the amino acid residue at position 10 of the amino acid sequence of SEQ ID NO: 4; or

c) in the amino acid residue at position 9 or 10 of the amino acid sequence of SEQ ID NO: 2 or SEQ ID NO: 11, or a variant thereof having at least 70%, more preferably at least 80%, even more preferably at least 90%, yet even more preferably at least 95%, most preferably at least 97%, 98% or 99% sequence identity to the amino acid sequence of SEQ ID NO: 2; or

d) in the amino acid residue at position 9 or 10 of the amino acid sequence of SEQ ID NO: 5.

In a preferred embodiment, the amino acid that is mutated at the respective position 9 or 10 is a lysine or an arginine or a valine.

In a preferred embodiment, the amino acid that is mutated at the respective position 9 or 10 is a lysine or an arginine.

In an embodiment, the amino acid residue at the respective position 9 or 10 may be modified into any amino acid except a lysine, an arginine or a histidine or a valine.

In an embodiment, the amino acid residue at the respective position 9 or 10 may be modified into any amino acid except a lysine, an arginine or a histidine.

In a preferred embodiment, the amino acid residue at the respective position 9 or 10 may be modified into an amino acid residue selected from the group consisting of serine, threonine, cysteine, methionine, tyrosine, glutamine, asparagine, glutamic acid and aspartic acid, and more preferably into a glutamic acid or an aspartic acid residue.

In a preferred embodiment, the amino acid residue at the respective position 9 or 10 may be modified into an amino acid residue selected from the group consisting of serine, threonine, cysteine, tyrosine, glutamine, asparagine, glutamic acid and aspartic acid, and more preferably into a glutamic acid or an aspartic acid residue.

In a preferred embodiment, the active mutation in sub c) is in the amino acid residue at position 9 of the amino acid sequence of SEQ ID NO: 2, or in a variant thereof having at least 70%, more preferably at least 80%, even more preferably at least 90%, yet even more preferably at least 95%, most preferably at least 97%, 98% or 99%, sequence identity to the amino acid sequence of SEQ ID NO: 2.

In a further preferred embodiment, active mutation in sub d) is in the amino acid residue at position 9 of the amino acid sequence of SEQ ID NO: 5.

In a further aspect, the present invention relates to a CenH3 protein of plant origin comprising the amino acid sequence of SEQ ID NO: 3 or a variant thereof having at least 70%, more preferably at least 80%, even more preferably at least 90%, yet even more preferably at least 95%, most preferably at least 97%, 98% or 99%, sequence identity to the amino acid sequence of SEQ ID NO: 3, in which the amino acid residue at position 9 is modified into any amino acid except a lysine, an arginine or a histidine.

In a preferred embodiment relating to the protein of plant origin comprising the amino acid sequence of SEQ ID NO: 3 or a variant thereof, the amino acid residue at position 9 is modified into an amino acid residue selected from the group consisting of serine, threonine, cysteine, tyrosine, glutamine, asparagine, glutamic acid and aspartic acid, and more preferably into a glutamic acid or an aspartic acid residue.

In an embodiment, the amino acid sequence of SEQ ID NO: 3 or a variant thereof as taught herein may be encoded by a polynucleotide comprising the nucleic acid sequence of SEQ ID NO: 6 or SEQ ID NO: 9 in which one or more nucleotides at positions 25-27 of the nucleic acid sequence of SEQ ID NO: 6 or at positions 25-27 of the nucleic acid sequence of SEQ ID NO: 9 are mutated to form a codon that translates into any amino acid except a lysine, an arginine or a histidine, preferably into a glutamic acid or an aspartic acid residue.

In a further aspect, the present invention relates to a CenH3 protein of plant origin comprising the amino acid sequence of SEQ ID NO: 8.

In an embodiment, the CenH3 protein of plant origin comprising the amino acid sequence of SEQ ID NO: 8 as taught herein may be encoded by a polynucleotide comprising the nucleic acid sequence of SEQ ID NO: 7 or SEQ ID NO: 10.

In a further aspect, the CenH3 protein of plant origin comprises the amino acid sequence of SEQ ID NO: 11 or a variant thereof having at least 70%, more preferably at least 80%, even more preferably at least 90%, yet even more preferably at least 95%, most preferably at least 97%, 98% or 99%, sequence identity to the amino acid sequence of SEQ ID NO: 11. Preferably said CenH3 protein comprises an active mutation at position 9, 12 or 22, or a combination thereof, of the amino acid sequence of SEQ ID NO: 11 or of the amino acid sequence of a variant having at least 70%, more preferably at least 80%, even more preferably at least 90%, yet even more preferably at least 95%, most preferably at least 97%, 98% or 99%, sequence identity to the amino acid sequence of SEQ ID NO: 11.

In an embodiment, the CenH3 protein of plant origin comprises the amino acid sequence of SEQ ID NO: 12 or a variant thereof having at least 70%, more preferably at least 80%, even more preferably at least 90%, yet even more preferably at least 95%, most preferably at least 97%, 98% or 99%, sequence identity to the amino acid sequence of SEQ ID NO: 12. Preferably, said CenH3 protein comprises an active mutation at position 9, 16 or 26, or a combination thereof, of the amino acid sequence of SEQ ID NO: 12 or of the amino acid sequence of a variant having at least 70%, more preferably at least 80%, even more preferably at least 90%, yet even more preferably at least 95%, most preferably at least 97%, 98% or 99%, sequence identity to the amino acid sequence of SEQ ID NO: 12.

Preferably, the indicated active mutation at position 9 of SEQ ID NO: 11 or 12, or variant thereof as defined herein, is a mutation wherein said amino acid residue is modified into an amino acid residue selected from the group consisting of methionine, serine and threonine, more preferably into methionine.

Preferably, the indicated active mutation at position 12 and/or 16 of SEQ ID NO: 11 or 12, or variant thereof as defined herein, is a mutation wherein said amino acid residue is modified into an amino acid residue selected from the group consisting of methionine, serine and threonine, more preferably into serine.

Preferably, the indicated active mutation at position 22 and/or 26 of SEQ ID NO: 11 or 12, or variant thereof as defined herein, is a mutation wherein said amino acid residue is modified into an amino acid residue selected from the group consisting of glycine, alanine, valine, leucine and isoleucine, more preferably into leucine.

In an embodiment, the CenH3 protein of plant origin comprises the amino acid sequence of SEQ ID NO: 13.

In an embodiment, the CenH3 protein of plant origin comprises the amino acid sequence of SEQ ID NO: 14.

In an embodiment, the CenH3 protein of plant origin comprises the amino acid sequence of SEQ ID NO: 15.

In an embodiment, the CenH3 protein of plant origin comprising the amino acid sequence of SEQ ID NO: 13 as taught herein may be encoded by a polynucleotide comprising the nucleic acid sequence of SEQ ID NO: 17 or SEQ ID NO: 21.

In an embodiment, the CenH3 protein of plant origin comprising the amino acid sequence of SEQ ID NO: 14 as taught herein may be encoded by a polynucleotide comprising the nucleic acid sequence of SEQ ID NO: 18 or SEQ ID NO: 22.

In an embodiment, the CenH3 protein of plant origin comprising the amino acid sequence of SEQ ID NO: 15 as taught herein may be encoded by a polynucleotide comprising the nucleic acid sequence of SEQ ID NO: 19 or SEQ ID NO: 23.

In an embodiment, when the CenH3 protein as taught herein, which is encoded by a CenH3 protein-encoding polynucleotide having an active mutation as taught herein, is present in a plant in the absence of its endogenous CenH3-encoding polynucleotide and/or endogenous CenH3 protein, it allows said plant to be viable, and allows generation of some haploid progeny, or progeny with aberrant ploidy, when said plant is crossed with a wild-type plant.

In an embodiment, the use of any one of the CenH3 proteins as taught herein causes at least 0.1, 0.5, 1 or 5% of the progeny generated to be haploid or to have an aberrant ploidy.

In an embodiment, the CenH3 protein as taught herein may be derived from an endogenous CenH3 protein by introducing mutations in the polynucleotide encoding said endogenous CenH3 protein using targeted nucleotide exchange or by applying an endonuclease.

In a preferred embodiment, the active mutation as taught herein is a point mutation.

In a further aspect, the present invention relates to a polynucleotide encoding any one of the CenH3 proteins as taught herein.

In an embodiment, the polynucleotide as taught herein is a polynucleotide comprising the nucleic acid sequence of SEQ ID NO: 6 or SEQ ID NO: 9, or a variant thereof having at least 70%, more preferably at least 80%, even more preferably at least 90%, yet even more preferably at least 95%, most preferably at least 97%, 98% or 99% sequence identity to the nucleic acid sequence of SEQ ID NO: 6 or SEQ ID NO: 9, in which one or more nucleotides at positions 25-27 of the nucleic acid sequence of SEQ ID NO: 6 or at positions 25-27 of the nucleic acid sequence of SEQ ID NO: 9 are modified such that the polynucleotide encodes a plant CenH3 protein in which the amino acid sequence of SEQ ID NO: 2 has an altered residue at position 9 or 10 or SEQ ID NO: 3, preferably has an altered residue at position 9.

In a preferred embodiment relating to the polynucleotide comprising the nucleic acid sequence of SEQ ID NO: 6 or SEQ ID NO: 9, or a variant thereof, the amino acid sequence of SEQ ID NO: 2 has an altered residue at position 9.

In an embodiment relating to the polynucleotide comprising the nucleic acid sequence of SEQ ID NO: 6 or SEQ ID NO: 9, or a variant thereof, the altered residue may be altered into any amino acid except a lysine, an arginine or a histidine.

In a preferred embodiment relating to the polynucleotide comprising the nucleic acid sequence of SEQ ID NO: 6 or SEQ ID NO: 9, or a variant thereof, the altered residue may be altered into an amino acid residue selected from the group consisting of serine, threonine, cysteine, tyrosine, glutamine, asparagine, glutamic acid and aspartic acid, and more preferably into a glutamic acid or an aspartic acid residue.

In a further aspect, the present invention relates to a polynucleotide comprising the nucleic acid sequence of SEQ ID NO: 7 or SEQ ID NO: 10.

In an embodiment, the polynucleotide as taught herein is a polynucleotide comprising the nucleic acid sequence of SEQ ID NO: 16 or SEQ ID NO: 20, or a variant thereof having at least 70%, more preferably at least 80%, even more preferably at least 90%, yet even more preferably at least 95%, most preferably at least 97%, 98% or 99% sequence identity to the nucleic acid sequence of SEQ ID NO: 16 or SEQ ID NO: 20, in which one or more nucleotides at positions 25-27, 46-48 and 76-78 of the nucleic acid sequence of SEQ ID NO: 16 or at positions 25-27, 46-48 and 76-78 of the nucleic acid sequence of SEQ ID NO: 20 are modified such that the polynucleotide encodes a plant CenH3 protein in which the amino acid sequence of SEQ ID NO: 11 has one or more altered residues at position 9, 12 and/or 22, or a plant CenH3 protein in which the amino of SEQ ID NO: 12 has one or more altered residues at position 9, 16 and/or 26.

In an embodiment relating to the polynucleotide comprising the nucleic acid sequence of SEQ ID NO: 16 or SEQ ID NO: 20, or a variant thereof, the altered residue at position 9 may be altered into methionine, serine or threonine, preferably into methionine; the altered residue at position 16 may be altered into methionine, serine or threonine, preferably into serine; and the altered residue at position 26 may be altered into glycine, alanine, valine, leucine and isoleucine, preferably into leucine.

In a further aspect, the present invention relates to a polynucleotide comprising the nucleic acid sequence of SEQ ID NO: 17 or SEQ ID NO: 21.

In a further aspect, the present invention relates to a polynucleotide comprising the nucleic acid sequence of SEQ ID NO: 18 or SEQ ID NO: 22.

In a further aspect, the present invention relates to a polynucleotide comprising the nucleic acid sequence of SEQ ID NO: 19 or SEQ ID NO: 23

In an embodiment, any one of the polynucleotides as taught herein are isolated.

In a further aspect, the present invention relates to a chimeric gene comprising any one of the polynucleotides as taught herein.

In a further aspect, the present invention relates to vector comprising any one of the polynucleotides as taught herein or the chimeric gene as taught herein.

In a further aspect, the present invention relates to host cell comprising any one of the polynucleotides as taught herein, the chimeric gene as taught herein, or the vector as taught herein.

In an embodiment the host cell as taught herein may be a plant cell, preferably a tomato plant cell or a tomato protoplast, preferably a Solanum plant cell or a Solanum protoplast, more preferably a Solanum lycopersicum plant cell or a Solanum lycopersicum protoplast.

In an embodiment the host cell as taught herein may be a plant cell, preferably a rice plant cell or a rice protoplast, preferably an Oryza plant cell or a Oryza protoplast, even more preferably an Oryza sativa plant cell or a Oryza sativa protoplast, even more preferably an Oryza sativa L. ssp. Japonica plant cell or Oryza sativa L. ssp. Japonica plant protoplast.

In a further aspect, the present invention relates to a plant comprising any one of the polynucleotides as taught herein, the chimeric gene as taught herein, or the vector as taught herein.

In an embodiment relating to the plants as taught herein, the endogenous CenH3 protein in said plant is not expressed.

In a preferred embodiment, the plant as taught herein may be a Solanum plant, more preferably a Solanum lycopersicum plant.

In a preferred embodiment, the plant as taught herein may be an Oryza plant, more preferably an Oryza sativa plant, even more preferably an Oryza sativa L. ssp. Japonica plant.

In an embodiment, the plant as taught herein may comprise two copies of an allele of a mutated polynucleotide encoding a CenH3 protein comprising an active mutation.

In a further aspect, the present invention relates to a method for making a plant as taught herein, said method comprising the steps of:

a) modifying a polynucleotide encoding an endogenous CenH3 protein within a plant cell to obtain a mutated polynucleotide encoding a CenH3 protein as taught herein;

b) selecting a plant cell comprising the mutated polynucleotide; and

c) optionally, regenerating a plant from said plant cell.

In a further aspect, the present invention relates to a method for making a plant as taught herein, said method comprising the steps of:

a) modifying an endogenous CenH3 protein-encoding polynucleotide within a plant cell to obtain a CenH3 protein-encoding polynucleotide having an active mutation in its N-terminal tail domain;

b) selecting a plant cell comprising the CenH3 protein-encoding polynucleotide having an active mutation; and

c) optionally, regenerating a plant from said plant cell.

In a further aspect, the present invention relates to a method for making a plant as taught herein, said method comprising the steps of:

a) transforming a plant cell with the polynucleotide as taught herein, the chimeric gene as taught herein, or the vector as taught herein;

b) selecting a plant cell comprising the polynucleotide as taught herein, the chimeric gene as taught herein, and/or the vector as taught herein; and

c) optionally, regenerating a plant from said plant cell.

In an embodiment, the methods as taught herein may further comprise the step of:
modifying said plant cell to prevent expression of endogenous CenH3 protein.

In an embodiment, the endogenous CenH3 protein-encoding polynucleotide within said plant cell is modified to prevent expression of endogenous CenH3 protein.

In a further aspect, the present invention relates to a method of generating a haploid plant, a plant with aberrant ploidy or a doubled haploid plant, said method comprising the steps of:

a) crossing a plant expressing an endogenous CenH3 protein to the plant as taught herein, wherein the plant as taught herein does not express an endogenous CenH3 protein at least in its reproductive parts and/or during embryonic development;

b) harvesting seed;

c) growing at least one seedling, plantlet or plant from said seed, and

d) selecting a haploid seedling, plantlet or plant; a seedling, plantlet or plant with aberrant ploidy; or a doubled haploid seedling, plantlet or plant.

In a further aspect, the present invention relates to a method of generating a doubled haploid plant, said method comprising the step of:
converting the haploid seedling, plantlet or plant obtained in step d) above into a doubled haploid plant.

In an embodiment, the conversion may be performed by treatment with colchicine.

In an embodiment, the plant expressing an endogenous CenH3 protein may be an F1 plant.

In an embodiment, the plant expressing an endogenous CenH3 protein may be a pollen parent of the cross.

In an embodiment, the plant expressing an endogenous CenH3 protein may be an ovule parent of the cross.

In an embodiment, the cross may be performed at a temperature in the range of about 24° C. to about 30° C.

In an embodiment, the methods as taught herein do not comprise sexually crossing the whole genomes of said plants.

In a further aspect, the present invention relates to the use of any one of the polynucleotides as taught herein for producing a haploid inducer line.

In a further aspect, the present invention relates to a Solanum lycopersicum plant or seed comprising the CenH3 protein of SEQ ID NO: 3, which comprises one or more active mutations in its N-terminal tail domain.

In an embodiment relating to the Solanum lycopersicum plant or seed as taught herein, the one or more active mutations are in the CenH3 motif block 1.

In an embodiment relating to the Solanum lycopersicum plant or seed as taught herein, the amino acid residue at position 9 may be modified into any amino acid except a lysine, an arginine or a histidine.

In a preferred embodiment relating to the Solanum lycopersicum plant or seed as taught herein, the amino acid residue at position 9 may be modified into an amino acid selected from the group consisting of serine, threonine, cysteine, tyrosine, glutamine, asparagine, glutamic acid and aspartic acid, and more preferably into a glutamic acid or an aspartic acid residue.

In an embodiment, the Solanum lycopersicum plant or seed as taught herein may comprise any one of the polynucleotides as taught herein, the chimeric gene as taught herein, or the vector as taught herein.

In a further aspect, the present invention relates to a Solanum lycopersicum plant or seed comprising a polynucleotide encoding a protein comprising the amino acid sequence of SEQ ID NO: 8.

In a further aspect, the present invention relates to a Solanum lycopersicum plant or seed comprising a polynucleotide comprising the nucleic acid sequence of SEQ ID NO: 7 or SEQ ID NO: 10.

In a further aspect, the present invention relates to a Solanum lycopersicum plant or seed comprising a polynucleotide that encodes a CenH3 protein as taught herein.

In an embodiment relating to the Solanum lycopersicum plant or seed as taught herein, the endogenous CenH3 protein is not expressed at least in the reproductive parts and/or during embryonic development.

In a further aspect, the present invention relates to the use of the Solanum lycopersicum plant as taught herein for producing a haploid Solanum lycopersicum plant.

In a further aspect, the present invention relates to use of the Solanum lycopersicum plant as taught herein for producing a doubled haploid Solanum lycopersicum plant.

In a further aspect, the present invention relates to a Oryza sativa, preferably Oryza sativa L. ssp. Japonica, plant or seed comprising the CenH3 protein of SEQ ID NO: 12, which comprises one or more active mutations in its N-terminal tail domain.

In an embodiment relating to the Oryza sativa, preferably Oryza sativa L. ssp. Japonica, plant or seed as taught herein, the one or more active mutations are in the CenH3 motif block 1, preferably at position 9, which may be modified into methionine, serine or threonine, preferably into methionine.

In an embodiment relating to the Oryza sativa, preferably Oryza sativa L. ssp. Japonica, plant or seed as taught herein, the one or more active mutations are in the CenH3 N-terminal tail domain, preferably at position 16, which may be modified into methionine, serine or threonine, preferably into serine.

In an embodiment relating to the Oryza sativa, preferably Oryza sativa L. ssp. Japonica, plant or seed as taught herein, the one or more active mutations are in the CenH3 N-terminal tail domain, preferably at position 26, which may be modified into glycine, alanine, valine, leucine and isoleucine, preferably into leucine.

In an embodiment, the Oryza sativa, preferably Oryza sativa L. ssp. Japonica, plant or seed as taught herein may comprise any one of the polynucleotides as taught herein, the chimeric gene as taught herein, or the vector as taught herein.

In a further aspect, the present invention relates to a Oryza sativa, preferably Oryza sativa L. ssp. Japonica, plant or seed comprising a polynucleotide encoding a protein comprising the amino acid sequence of SEQ ID NO: 13, SEQ ID NO: 14 or SEQ ID NO: 15.

In a further aspect, the present invention relates to a Oryza sativa, preferably Oryza sativa L. ssp. Japonica, plant or seed comprising a polynucleotide comprising the nucleic acid sequence of SEQ ID NO: 17 or SEQ ID NO: 21.

In a further aspect, the present invention relates to a Oryza sativa, preferably Oryza sativa L. ssp. Japonica, plant or seed comprising a polynucleotide comprising the nucleic acid sequence of SEQ ID NO: 18 or SEQ ID NO: 22.

In a further aspect, the present invention relates to a Oryza sativa, preferably Oryza sativa L. ssp. Japonica, plant or seed comprising a polynucleotide comprising the nucleic acid sequence of SEQ ID NO: 19 or SEQ ID NO: 23.

In a further aspect, the present invention relates to a Oryza sativa, preferably Oryza sativa L. ssp. Japonica, plant or seed comprising a polynucleotide that encodes a CenH3 protein as taught herein.

In an embodiment relating to the Oryza sativa, preferably Oryza sativa L. ssp. Japonica, plant or seed as taught herein, the endogenous CenH3 protein is not expressed at least in the reproductive parts and/or during embryonic development.

In a further aspect, the present invention relates to the use of the Oryza sativa, preferably Oryza sativa L. ssp. Japonica, plant as taught herein for producing a haploid Oryza sativa, preferably Oryza sativa L. ssp. Japonica, plant.

In a further aspect, the present invention relates to use of the Oryza sativa, preferably Oryza sativa L. ssp. Japonica, plant as taught herein for producing a doubled haploid Oryza sativa, preferably Oryza sativa L. ssp. Japonica, plant.

In a further aspect, the present invention relates to a method of generating a haploid or doubled haploid plant, said method comprising identifying a plant expressing an endogenous CenH3 protein and a plant as taught herein, wherein the plant as taught herein does not express endogenous CenH3 protein at least in its reproductive parts and/or during embryonic development.

In an embodiment, the method as taught herein does not comprise sexually crossing the whole genomes of said plants.

### Definitions

The term ‘centromere-specific variant of histone H3 protein (abbreviated as ‘CenH3’ protein’), as used herein, refers to a protein that is a member of the kinetochore complex.

CenH3 protein is also known as ‘CENP-A’ protein. The kinetochore complex is located on chromatids where the spindle fibers attach during cell division to pull sister chromatids apart. CenH3 proteins belong to a well-characterized class of proteins that are variants of H3 histone proteins. These proteins are essential for proper formation and function of the kinetochore, and help the kinetochore associate with DNA. Cells that are deficient in CenH3 fail to localize kinetochore proteins on chromatids and show strong chromosome segregation defects (i.e. all chromosomes from the plant expressing the deficient CenH3 protein are eliminated or lost, leading to a change in the ploidy of somatic cells (e.g. reduction in the number of chromosome set such as diploid to haploid)). Therefore, CenH3 proteins have been subject to intensive research for their potential use in doubled haploid production system. CenH3 proteins are characterized by a variable tail domain (also referred to as ‘N-terminal domain’ or ‘N-terminal tail domain’) and a conserved histone fold domain (also referred to as C-terminal domain) made up of three alpha-helical regions connected by loop sections. The CenH3 histone fold domain is relatively well conserved between CenH3 proteins from different species. The histone fold domain is located at the carboxyl terminus of an endogenous CenH3 protein. In contrast to the histone-fold domain, the N-terminal tail domain of CenH3 is highly variable even between closely related species.

The term ‘consensus sequence’ as used herein refers to the calculated order of most frequent residues, either nucleotide or amino acid, found at each position in a sequence alignment. It represents the results of multiple sequence alignments (e.g. CenH3 sequences) in which related sequences (e.g. sequences of the N-tail domain of CenH3 proteins taken from different plants) are compared to each other and similar sequence motifs are calculated (e.g. using motif search program (e.g. MEME)). The skilled person is well-acquainted with the concept of ‘consensus sequence’ as well as with methodologies suitable for identifying consensus sequences in proteins across different plants (e.g. crop plants).

The term ‘haploid inducer line’, as used herein refers to a plant line which differs in at least one single nucleotide polymorphism from the non-inducer line. When an haploid inducer line is crossed, either used as female or as pollen donor, it results in uniparental genome elimination of the haploid inducer line's genome.

A CenH3-encoding polynucleotide having one or more active mutations' refers to a non-endogenous or endogenous mutated CenH3-encoding polynucleotide that encodes a CenH3 protein having one or more active mutations, which, when present in a plant in the absence of its endogenous CenH3-encoding polynucleotide and/or endogenous CenH3 protein, allows said plant to be viable, and allows generation of haploid progeny, or progeny with aberrant ploidy, when said plant is crossed with a wild-type plant, preferably a wild-type plant of the same species. The plant comprising a CenH3-encoding polynucleotide having one or more active mutations may be referred to as a ‘modified plant’. The percentage of haploid progeny or progeny with aberrant ploidy that is generated upon crossing with a wild-type plant can, for instance, be at least 0.1, 0.5, 1, 5, 10, 20% or more. A mutation that causes a transition from the endogenous CenH3-encoding polynucleotide to a CenH3-encoding polynucleotide having one or more active mutations is herein referred to as an ‘active mutation’. An active mutation in a CenH3 protein context may result, among other things, in reduced centromere loading, a less functional CenH3 protein and/or a reduced functionality in the separation of chromosomes during cell division. One or more active mutations may be introduced into the CenH3-encoding polynucleotide by any of several methods well-known to the skilled person, for example, by random mutagenesis, such as induced by treatment of seeds or plant cells with chemicals or radiation, targeted mutagenesis, the application of endonucleases, by generation of partial or complete protein domain deletions, or by fusion with heterologous sequences.

A ‘CenH3 protein having one or more active mutations’ is encoded by a CenH3-encoding polynucleotide having one or more active mutations. The endogenous CenH3-encoding polynucleotide encodes the endogenous CenH3 protein.

A plant may be made to lack the endogenous CenH3-encoding polynucleotide by knocking out or inactivating said endogenous CenH3-encoding polynucleotide. Alternatively, said endogenous CenH3-encoding polynucleotide may be modified to encode an inactive or non-functional CenH3 protein.

The modified plant comprising the CenH3-encoding polynucleotide having one or more active mutations as taught herein may be crossed to a wild-type plant either as a pollen parent or as an ovule parent. In an embodiment, a CenH3 protein having one or more active mutations may comprise 1, 2, 3, 4, 5, 6, 7, 8, 9, 10, 12, 15, 20 or more amino acid changes relative to the endogenous CenH3 protein. In an embodiment, a CenH3-encoding polynucleotide having one or more active mutations has 70, 75, 80, 85, 90, 95, 96, 97, 98, 99, 99.5% sequence identity to the endogenous CenH3-encoding polynucleotide, preferably over the full length.

The skilled person would readily be able to ascertain whether or not a modified plant as taught herein comprises one or more active mutations. For example, the skilled person may make use of predictive tools such as SIFT (Kumar P, Henikoff S, Ng PC. (2009) Predicting the effects of coding non-synonymous variants on protein function using the SIFT algorithm. Nat Protoc; 4(7):1073-81. doi:10.1038/nprot.2009.86) to propose such active mutation. The one or more active mutations may then be made in a plant, and expression of endogenous CenH3 protein in said plant should be knocked out. The plant may be considered to comprise one or more active mutations when the percentage of haploid progeny or progeny with aberrant ploidy that is generated upon crossing with a wild-type plant is at least 0.1, 0.5, 1, 5, 10, 20% or more.

Crossing a plant that lacks an endogenous CenH3-encoding polynucleotide or that lacks expression of endogenous CenH3 protein and that expresses a CenH3 protein having one or more active mutations either as a pollen or as an ovule parent with a plant that expresses an endogenous CenH3 protein results in a certain percentage (for instance at least 0.1, 0.5, 1, 5, 10, 20% or more) of progeny that is haploid or shows aberrant ploidy. Such a plant comprises only chromosomes of the parent that expresses the endogenous CenH3 protein, and no chromosomes of the plant expressing the CenH3 protein having one or more active mutation.

Two plants that are crossed may be of the same genus or of the same species. The crossing methods as taught herein do not comprise sexually crossing the whole genomes of said plants. Instead, one set of chromosomes is eliminated.

The term ‘aberrant ploidy’ as used herein refers to a situation where a cell comprises an aberrant or abnormal number of sets of chromosomes. For instance, a cell having one or three sets of chromosomes per cell when the usual number is two is a cell having aberrant ploidy. In the present invention, the active mutant CenH3 proteins and methods using them, as taught herein, can be used to generate mutant plants having aberrant ploidy, e.g. to generate haploid plants while the non-mutant plant is diploid. The haploid plants can be used to accelerate breeding programs to create homozygous lines and obviate the need for inbreeding.

The term ‘endogenous’ as used in the context of the present invention in combination with protein or gene means that said protein or gene originates from the plant in which it is still contained. Often an endogenous gene will be present in its normal genetic context in the plant.

The term ‘uniparental genome elimination’ as used herein refers to the effect of losing all the genetic information, meaning all chromosomes, of one parent after a cross irrespective of the direction of the cross. This occurs in such way that the offspring of such cross will only contain chromosomes of the non-eliminated parental genome. The genome which is eliminated always has the origin in the haploid inducer parent.

The terms ‘polynucleotide’ and ‘nucleic acid’ are used interchangeably herein.

A ‘chimeric gene’ (or recombinant gene) refers to any gene, which is not normally found in nature in a species, in particular a gene in which one or more parts of the nucleic acid sequence are present that are not associated with each other in nature. For example the promoter is not associated in nature with part or all of the transcribed region or with another regulatory region. The term ‘chimeric gene’ is understood to include expression constructs in which a promoter or transcription regulatory sequence is operably linked to one or more coding sequences or to an antisense (reverse complement of the sense strand) or inverted repeat sequence (sense and antisense, whereby the RNA transcript forms double stranded RNA upon transcription).

‘Sequence identity’ and ‘sequence similarity’ can be determined by alignment of two peptide or two nucleotide sequences using global or local alignment algorithms. Sequences may then be referred to as ‘substantially identical’ or ‘essentially similar’ when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length, maximizing the number of matches and minimises the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or EmbossWin version 2.10.0 (using the program ‘needle’). Alternatively percent similarity or identity may be determined by searching against databases, using algorithms such as FASTA, BLAST, etc.

A ‘host cell’ or a ‘recombinant host cell’ or ‘transformed cell’ are terms referring to a new individual cell (or organism) arising as a result of introduction of at least one nucleic acid molecule, especially comprising a chimeric gene encoding a desired protein. The host cell is preferably a plant cell or a bacterial cell. The host cell may contain the nucleic acid molecule or chimeric gene as an extra-chromosomally (episomal) replicating molecule, or more preferably, comprises the nucleic acid molecule or chimeric gene integrated in the nuclear or plastid genome of the host cell.

As used herein, the term ‘plant’ includes plant cells, plant tissues or organs, plant protoplasts, plant cell tissue cultures from which plants can be regenerated, plant calli, plant cell clumps, and plant cells that are intact in plants, or parts of plants, such as embryos, pollen, ovules, fruit (e.g. harvested tomatoes), flowers, leaves, seeds, roots, root tips and the like.

The term ‘doubled haploid plant’ as used herein refers to a genotype formed when haploid cells undergo chromosome doubling. Artificial production of doubled haploids is important in plant breeding. Doubled haploids can be produced in vivo or in vitro. Haploid embryos are produced in vivo by parthenogenesis, pseudogamy, or chromosome elimination after wide crossing. A wide variety of in vitro methods are known for generating doubled haploid organisms from haploid organisms. The skilled person is well-acquainted with such methods. A non-limiting example of a method for generating doubled haploid in vitro consist of treating somatic haploid cells, haploid embryos, haploid seeds, or haploid plants produced from haploid seeds with a chromosome doubling agent such as colchicine. In the present invention, homozygous double haploid plants can be regenerated from haploid cells by contacting the haploid cells with chromosome doubling agents, such as colchicine, anti-microtubule herbicides, or nitrous oxide to create homozygous doubled haploid cells. Methods of chromosome doubling are disclosed in, for example, U.S. Pat. Nos. 5,770,788; 7,135,615, and US Patent Publication No. 2004/0210959 and 2005/0289673; Antoine-Michard, S. et al., Plant Cell, Tissue Organ Cult., Cordrecht, the Netherlands, Kluwer Academic Publishers 48(3):203-207 (1997); Kato, A., Maize Genetics Cooperation Newsletter 1997, 36-37; and Wan, Y. et al., Trends Genetics 77: 889-892 (1989). Wan, Y. et al., Trends Genetics 81: 205-211 (1991), the disclosures of which are incorporated herein by reference. Double haploid plants can be further crossed to other plants to generate FI, F2, or subsequent generations of plants with desired traits. Conventional inbreeding procedures take six generations to achieve approximately complete homozygosity, whereas doubled haploidy achieves it in one generation.

In the context of the present invention, the use of the term ‘wild type plant’ refers to a plant which does not carry a mutant CenH3 protein or gene (i.e. does not comprise one or more active mutations as taught herein) and which endogenously expresses or produces functional CenH3 genes and proteins.

In this document and in its claims, the verb ‘to comprise’ and its conjugations is used in its non-limiting sense to mean that items following the word are included, but items not specifically mentioned are not excluded. It encompasses the verbs ‘to essentially consist of’ and ‘to consist of’.

In addition, reference to an element by the indefinite article ‘a’ or ‘an’ does not exclude the possibility that more than one of the element is present, unless the context clearly requires that there be one and only one of the elements. The indefinite article ‘a’ or ‘an’ thus usually means ‘at least one’. It is further understood that, when referring to ‘sequences’ herein, generally the actual physical molecules with a certain sequence of subunits (e.g. amino acids) are referred to.

### DETAILED DESCRIPTION OF THE INVENTION

The present inventors found that elimination or disruption of an endogenous CenH3 in combination with expression of a non-endogenous CenH3 protein having one or more active specific mutations (e.g. point mutation) in a plant resulted in a plant that has useful properties for breeding. It was found that such plant can function as a haploid inducer line. When such haploid inducer line is crossed with a plant having an endogenous CenH3 protein, a portion of the resulting progeny lacks the chromosomes from the haploid inducer line, thereby allowing the production of haploid progeny or progeny with aberrant ploidy (i.e. abnormal number of chromosome set in somatic cells). Haploid plants are useful for improving breeding.

Equal distribution of DNA in mitosis requires the assembly of a large proteinaceous ensemble onto the centromeric DNA, called the kinetochore. Kinetochores are multi-subunit complexes that assemble on centromeres to bind spindle microtubules and promote faithful chromosome segregation during cell division. A 16-subunit complex named the constitutive centromere-associated network (CCAN) creates the centromere-kinetochore interface.

CenH3, a CCAN subunit, is crucial for kinetochore assembly because it links centromeres with the microtubule-binding interface of kinetochores. The exact role of CenH3 in CCAN organization is not yet fully understood. When CenH3 is depleted or absent or dysfunctional, the proper formation of both centromeres and kinetochores is prevented.

More specifically, the present inventors surprisingly found that plants with a modified CenH3 protein, i.e. comprising one or more active specific mutations (e.g. point mutation) in the N-terminal tail domain as taught herein, are able to induce haploid offspring after a cross to or with a wild type plant lacking these particular mutations in CenH3 protein.

It was thought that any number of active mutations (e.g. point mutations) can be introduced into a CenH3 protein or a gene encoding a CenH3 protein to generate an active mutant CenH3 protein capable of generating haploid plants. However, this is not the case since not all mutations in the CenH3 protein or gene have turned out to be active mutations, i.e. result in the production of a haploid plant. This is particularly true for mutations or alterations in the N-terminal tail domain of CenH3 proteins or genes encoding CenH3 proteins. So far, inactivation of the whole N-terminal tail of CenH3 proteins (e.g. using a tailswap) in a plant has been used to cause uniparental genome elimination for the purpose of generating haploid plants. It was not known whether one active specific mutation (e.g. point mutations) in the N-terminal tail domain of CenH3 protein or CenH3 gene encoding it would be sufficient to generate a haploid plant. That is because the N-terminal tail domain of CenH3 proteins is highly variable between species (even closely related ones) and there were no indications as to which part of the N-terminal tail or what type of mutation would produce the desired effect, i.e. generate haploid plants. Overall, this made it difficult, labour-intensive and time-consuming to identify and/or predict whether a given mutation, particularly a point mutation causing a single change in amino acid, will have any effect in a given plant, i.e. result in the production of a haploid plant.

The present inventors have found that the introduction of a specific point mutation in a specific region of the N-tail domain of the CenH3 protein was sufficient to generate a haploid plant. Specifically, the inventors found that a plant comprising such modified CenH3 protein and lacking a functional (e.g., endogenous) CenH3 protein, can be used as an ‘haploid inducer plant’ to effectively cause the elimination of one parental genome to generate haploid progeny by crossing the haploid inducer plant with a plant comprising an endogenous CenH3 protein.

In other words, the present inventors found a reliable, efficient and rapid way to convert a natural diploid plant cell into a haploid cell, simply by introducing one or more active specific mutations (e.g. point mutations) as taught herein, causing a change in a single amino acid in the CenH3 protein as taught herein. The method of the invention is applicable to a wide variety of crop plants since the region in the N-tail domain of the CenH3 used to incorporate the one or more active specific mutations as taught herein is universal across all plants.

CenH3 Proteins Having an Active Mutation

In a first aspect, the present invention relates to a CenH3 protein of plant origin comprising one or more active mutations in its N-terminal tail domain, e.g. point mutation causing a change in a single amino acid.

When a plant that expresses such CenH3 protein having one or more active mutations and lacks expression of, or has suppressed expression of, endogenous CenH3 protein, is crossed to a wild type plant expressing endogenous CenH3 protein or functional CenH3 protein, haploid plants (or plant with aberrant ploidy) are formed at relatively high frequency. CenH3 proteins having one or more active mutations in the N-terminal tail domain, as taught herein, can be created by a variety of means known to the skilled person. These include, without limitation, random mutagenesis, single or multiple amino acid targeted mutagenesis, generation of complete or partial protein domain deletions, fusion with heterologous amino acid sequences, and the like. Typically, in such plant, the polynucleotide encoding endogenous CenH3 protein will be knocked out or inactivated. Haploid plants are formed at a more than normal frequency, such as at least 0.1, 0.5, 1, 5, 10, 20% or more. CenH3 proteins having one or more active mutations and variants thereof can, for example, be tested by recombinant expression of the CenH3 protein having one or more active mutations in a plant lacking endogenous CenH3 protein, crossing the transgenic plant to a plant expressing endogenous CenH3 protein or functional CenH3 protein, and then screening for the production of haploid progeny.

The plant CenH3 proteins and variants thereof, as taught herein, may be any plant CenH3 proteins. In a preferred embodiment, plant CenH3 proteins belong to the Solanaceae family, more preferably to the genus Solanum, even more preferably to the species Solanum lycopersicum.

In an embodiment, the CenH3 proteins as taught herein may comprise one or more active specific mutations (e.g. point mutation causing a change in a single amino acid) which are located in the plant consensus CenH3 motif block 1 domain in the N-terminal tail domain. The term ‘plant CenH3 consensus motif block 1 domain protein sequence’, as used herein, refers to a modular pattern of sequence conservation (i.e. consensus sequence) located in the N-tail domain of CenH3 proteins that is highly conserved among all plant species. Its amino acid sequence is shown in SEQ ID NO: 4. Despite hyper-variability both in the amino acid sequence and length of the N-tail domain of CenH3 gene and protein, seven stretches of conserved protein sequences (referred to as ‘motif block 1 to 7’) were identified in the N-terminal tails of CenH3 proteins of various plants (Maheshwari et al (2015) PLOS Genetics, DOI:10.1371/journal.pgen.1004970, pages 1-20). Motif block 1 has been identified in nearly all plant CenH3 proteins. Therefore, in an embodiment, the plant CenH3 consensus motif block 1 domain protein sequence (SEQ ID NO: 4) can be used as a plant CenH3 DH-inducer motif block 1 domain protein sequence. The term ‘plant CenH3 DH-inducer motif block 1 domain protein sequence’, as used herein, refers to plant CenH3 consensus motif block 1 domain protein sequence’ as taught above comprising one or more active mutations (e.g. point mutation) in the amino acid sequence of SEQ ID NO: 4. When present in a plant, the plant CenH3 DH-inducer motif block 1 domain protein sequence with one or more active mutations allows the generation of some haploid progeny, or progeny with aberrant ploidy, when said plant is crossed with a wild-type plant, preferably a wild-type plant of the same species.

In an embodiment, the CenH3 proteins as taught herein may comprise one or more active specific mutations (e.g. point mutation causing a change in a single amino acid) in the plant CenH3 consensus protein sequence. The term ‘plant CenH3 consensus protein sequence’ as used herein refers to a specific region of the CenH3 protein that is highly conserved among all plant species, and its amino acid sequence is shown in SEQ ID NO: 1. Therefore, in an embodiment, the plant CenH3 consensus protein sequence (SEQ ID NO: 1) can be used as a plant CenH3 DH-inducer protein sequence. The term ‘plant CenH3 DH-inducer protein sequence’ as used herein refers to the plant CenH3 consensus protein sequence as taught above comprising one or more active mutations (e.g. point mutation) in the amino acid sequence of SEQ ID NO: 1. When present in a plant, the plant CenH3 DH-inducer protein sequence with one or more active mutations allows the generation of haploid progeny, or progeny with aberrant ploidy, when said plant is crossed with a wild-type plant, preferably a wild-type plant of the same species.

In an embodiment, the CenH3 proteins as taught herein may comprise one or more active specific mutations (e.g. point mutation causing a change in a single amino acid) which are located in Solanaceae CenH3 consensus protein sequence. The term ‘Solanaceae CenH3 consensus protein sequence’, as used herein, refers to the CenH3 protein from a species belonging to the Solanaceae plant family that is highly conserved among Solanaceae species, (e.g. Solanum lycopersicum, Nicotiana tabacum, Nicotiana tomentosiformis, Capsicum annuum, Solanum tuberosum and Solanum frutescence), and its amino acid sequence is shown in SEQ ID NO: 2. Therefore, in an embodiment, the Solanaceae CenH3 consensus protein sequence (SEQ ID NO: 2) can be used as a Solanaceae CenH3 DH-inducer protein sequence. The term ‘Solanaceae CenH3 DH-inducer protein sequence’, as used herein, refers to the Solanaceae CenH3 consensus protein sequence as taught above comprising one or more active mutations (e.g. point mutation) in the amino acid sequence of SEQ ID NO: 2. When present in a plant, the Solanaceae CenH3 DH-inducer protein sequence with one or more active mutations allows the generation of haploid progeny, or progeny with aberrant ploidy, when said plant is crossed with a wild-type plant, preferably a wild-type plant of the same species.

In an embodiment, the CenH3 proteins as taught herein may comprise one or more active specific mutations (e.g. point mutation causing a change in a single amino acid) which are located in the Solanaceae CenH3 consensus motif block 1 protein sequence. The term ‘Solanaceae CenH3 consensus motif block 1 protein sequence’, as used herein, refers to a modular pattern of sequence conservation (i.e. consensus sequence) located in the N-tail domain of CenH3 proteins from species belonging to the Solanum plant genus. It is highly conserved among Solanum species (e.g. Solanum lycopersicum, Nicotiana tabacum, Nicotiana tomentosiformis, Capsicum annuum, Solanum tuberosum and Solanum frutescence), and its amino acid sequence is shown as SEQ ID NO: 5. Therefore, in an embodiment, the Solanaceae CenH3 consensus motif block 1 protein sequence (SEQ ID NO: 5) can be used as a Solanaceae CenH3 DH-inducer motif block 1 protein sequence. The term ‘Solanaceae CenH3 DH-inducer motif block 1 protein sequence’, as used herein, refers to the Solanaceae CenH3 consensus motif block 1 protein sequence as taught above comprising one or more active mutations (e.g. point mutations) in the amino acid sequence of SEQ ID NO: 5. When present in a plant, the ‘Solanaceae CenH3 DH-inducer motif block 1 protein sequence with one or more active mutations allows the generation of some haploid progeny, or progeny with aberrant ploidy, when said plant is crossed with a wild-type plant, preferably a wild-type plant of the same species.

In an embodiment, the CenH3 proteins as taught herein may comprise one or more active mutations, which are present:

a) in a protein comprising the amino acid sequence of SEQ ID NO: 1, or in a variant thereof having at least 70%, more preferably at least 80%, even more preferably at least 90%, yet even more preferably at least 95%, most preferably at least 97%, 98% or 99% sequence identity to the amino acid sequence of SEQ ID NO: 1;

b) in a protein comprising the amino acid sequence of SEQ ID NO: 4;

c) in a protein comprising the amino acid sequence of SEQ ID NO: 2, or in a variant thereof having at least 70%, more preferably at least 80%, even more preferably at least 90%, yet even more preferably at least 95%, most preferably at least 97%, 98% or 99% sequence identity to the amino acid sequence of SEQ ID NO: 2; or

d) in a protein comprising the amino acid sequence of SEQ ID NO: 5.

In a preferred embodiment, CenH3 proteins as taught herein may comprise one active mutation that consists of:

a) an active mutation in the amino acid residue at position 10 of the amino acid sequence of SEQ ID NO: 1, or in a variant thereof having at least 70%, more preferably at least 80%, even more preferably at least 90%, yet even more preferably at least 95%, most preferably at least 97%, 98% or 99% sequence identity to the amino acid sequence of SEQ ID NO: 1;

b) an active mutation in the amino acid residue at position 10 of the amino acid sequence of SEQ ID NO: 4;

c) an active mutation in the amino acid residue at position 9 or 10 of the amino acid sequence of SEQ ID NO: 2, or a variant thereof having at least 70%, more preferably at least 80%, even more preferably at least 90%, yet even more preferably at least 95%, most preferably at least 97%, 98% or 99% sequence identity to the amino acid sequence of SEQ ID NO: 2; or

d) an active mutation in the amino acid residue at position 9 or 10 of the amino acid sequence of SEQ ID NO: 5.

In a preferred embodiment, the active mutation in option c) is in the amino acid residue at position 9 of the amino acid sequence of SEQ ID NO: 2, or a variant thereof having at least 70%, more preferably at least 80%, even more preferably at least 90%, yet even more preferably at least 95%, most preferably at least 97%, 98% or 99% sequence identity to the amino acid sequence of SEQ ID NO: 2.

In a further preferred embodiment, the active mutation in option d) is in the amino acid residue at position 9 of the amino acid sequence of SEQ ID NO: 5.

In an embodiment, the amino acid that is mutated at the respective position 9 or 10 as taught above may be a lysine or an arginine.

In an embodiment, the amino acid residue at the respective position 9 or 10 as taught above may be modified into any amino acids except a lysine, an arginine or a histidine.

In a preferred embodiment, the amino acid residue at the respective position 9 or 10 as taught above may be modified into an amino acid residue selected from the group consisting of serine, threonine, cysteine, tyrosine, glutamine, asparagine, glutamic acid and aspartic acid.

In a further preferred embodiment, the amino acid residue at the respective position 9 or 10 as taught above may be modified into a glutamic acid or an aspartic acid residue. Such modification changes the charge (from positively to negatively) on the amino acid residue at this position and was found to be a highly suitable active mutation, i.e., suitable for the generation of haploid or doubled haploid plants.

In an embodiment, the CenH3 protein of plant origin as taught herein may comprise the amino acid sequence of SEQ ID NO: 3 (i.e. Solanum lycopersicum CenH3 protein amino acid sequence) or a variant thereof having at least 70%, more preferably at least 80%, even more preferably at least 90%, yet even more preferably at least 95%, most preferably at least 97%, 98% or 99%, sequence identity to the amino acid sequence of SEQ ID NO: 3, in which the amino acid residue at position 9 is modified into any amino acid except a lysine, an arginine or a histidine. Such protein is also denominated herein as a “Solanum lycopersicum CenH3 mutant”.

In a preferred embodiment, the amino acid residue at position 9 is modified into an amino acid residue selected from the group consisting of serine, threonine, cysteine, tyrosine, glutamine, asparagine, glutamic acid and aspartic acid.

In a further preferred embodiment, the amino acid residue at position 9 is modified into an amino acid residue selected from a glutamic acid or an aspartic acid residue. Such modification changes the charge (from positively to negatively) on the amino acid residue at this position and was found to be a highly suitable active mutation, i.e., suitable for the generation of haploid or doubled haploid plants.

In an embodiment, the CenH3 protein comprising the amino acid sequence of SEQ ID NO: 3 or variants thereof as taught herein and in which the amino acid residue at position 9 is modified into a different amino acid as taught above, may be encoded by a polynucleotide comprising the nucleic acid sequence of SEQ ID NO: 6 or SEQ ID NO: 9 in which one or more nucleotides at positions 25-27 of the nucleic acid sequence of SEQ ID NO: 6 or at positions 25-27 of the nucleic acid sequence of SEQ ID NO: 9 are mutated to form a codon that translates into any amino acid except a lysine, an arginine or a histidine, preferably into a glutamic acid or an aspartic acid residue.

In an embodiment, the percentage in sequence identity to the amino acid sequences of SEQ ID NO: 1, SEQ ID NO: 2, SEQ ID NO: 3, SEQ ID NO: 4, SEQ ID NO: 5 or SEQ ID NO: 12 is preferably over the entire length. Amino acid sequence identity may be determined by any known methods, for instance by pairwise alignment using the Needleman and Wunsch algorithm and GAP default parameters as defined above.

In an embodiment, the percentage in sequence identity to the nucleotide sequences of SEQ ID NO: 6, SEQ ID NO: 9, SEQ ID NO: 16 or SEQ ID NO: 20 is preferably over the entire length. Nucleotide sequence identity may be determined by any known methods, for instance by pairwise alignment using the Needleman and Wunsch algorithm and GAP default parameters as defined above.

In an embodiment, the CenH3 protein of plant origin as taught herein may comprise the amino acid sequence of SEQ ID NO: 8.

The term ‘Solanum lycopersicum CenH3_K9E amino acid sequence’ as used herein refers to a mutant Solanum lycopersicum CenH3 protein amino acid sequence comprising a single point mutation in the amino acid residue at position 9 of SEQ ID NO: 8, which causes the modification of a lysine to a glutamate. The present inventors found that the mutant Solanum lycopersicum CenH3_K9E amino acid sequence (SEQ ID NO: 8) is particularly advantageous for use as a Solanum lycopersicum CenH3_K9E DH-inducer protein sequence, e.g. in plant breeding programs. It was found that when present in a plant, the Solanum lycopersicum CenH3_K9E DH-inducer protein sequence allows the generation of haploid progeny, or progeny with aberrant ploidy, when said plant is crossed with a wild-type plant, preferably a wild-type plant of the same species, at a particularly high rate than what achieved by traditional methods.

In an embodiment, Solanum lycopersicum CenH3_K9E amino acid sequence (SEQ ID NO: 8), when present in a plant and when said plant is crossed with a wild-type plant, preferably a wild-type plant of the same species, at least 0.1, 0.5, 1 or 5% of the progeny generated is haploid or has aberrant ploidy.

The Solanum lycopersicum CenH3_K9E CenH3 protein as taught above may be encoded by the nucleic acid sequence of SEQ ID NO: 7 or SEQ ID NO: 10.

In an embodiment, the CenH3 proteins as taught herein may comprise one or more active specific mutations (e.g. point mutation causing a change in a single amino acid) which are located in the monocotyledon CenH3 consensus protein sequence. The term ‘monocotyledon CenH3 consensus protein sequence’, as used herein, refers to the CenH3 protein from a species belonging to the monocotyledon plant family that is highly conserved among monocotyledon species, (e.g. Allium cepa, Allium fistulosum, Allium sativum, Allium tuberosum, Hordeum bulbosum, Hordeum vulgare, Luzula nivea, Oryza sativa, Panicum virgatum, Saccharum officinarum, Setaria italic, Sorghum bicolor, Zea mays), and its amino acid sequence is shown in SEQ ID NO: 11. Therefore, in an embodiment, the monocotyledon CenH3 consensus protein sequence (SEQ ID NO: 11) can be used as a monocotyledon CenH3 DH-inducer protein sequence. The term ‘monocotyledon CenH3 DH-inducer protein sequence’, as used herein, refers to the monocotyledon CenH3 consensus protein sequence as taught above comprising one or more active mutations (e.g. point mutation) in the amino acid sequence of SEQ ID NO: 11. When present in a plant, the monocotyledon CenH3 DH-inducer protein sequence with one or more active mutations allows the generation of haploid progeny, or progeny with aberrant ploidy, when said plant is crossed with a wild-type plant, preferably a wild-type plant of the same species.

In an embodiment, the plant CenH3 proteins and variants thereof may be monocotyledon CenH3 proteins and variants thereof, preferably monocotyledon CenH3 proteins that belong to the Poaceae family, more preferably to the genus Oryza, even more preferably to the species Oryza sativa, even more preferably of the subspecies Oryza sativa L. ssp. japonica.

In an embodiment, the CenH3 proteins as taught herein may comprise one or more active mutations, which is present:

a) in a protein comprising the amino acid sequence of SEQ ID NO: 11, or in a variant thereof having at least 70%, more preferably at least 80%, even more preferably at least 90%, yet even more preferably at least 95%, most preferably at least 97%, 98% or 99% sequence identity to the amino acid sequence of SEQ ID NO: 11; or

b) in a protein comprising the amino acid sequence of SEQ ID NO: 4.

In a preferred embodiment, CenH3 proteins as taught herein may comprise one active mutation that consists of:

a) an active mutation in the amino acid residue at position 9 of the amino acid sequence of SEQ ID NO: 11, or in a variant thereof having at least 70%, more preferably at least 80%, even more preferably at least 90%, yet even more preferably at least 95%, most preferably at least 97%, 98% or 99% sequence identity to the amino acid sequence of SEQ ID NO: 11;

b) an active mutation in the amino acid residue at position 12 of the amino acid sequence of SEQ ID NO: 11, or in a variant thereof having at least 70%, more preferably at least 80%, even more preferably at least 90%, yet even more preferably at least 95%, most preferably at least 97%, 98% or 99% sequence identity to the amino acid sequence of SEQ ID NO: 11;

c) an active mutation in the amino acid residue at position 22 of the amino acid sequence of SEQ ID NO: 11, or in a variant thereof having at least 70%, more preferably at least 80%, even more preferably at least 90%, yet even more preferably at least 95%, most preferably at least 97%, 98% or 99% sequence identity to the amino acid sequence of SEQ ID NO: 11; or

d) an active mutation in the amino acid residue at position 9 of the amino acid sequence of SEQ ID NO: 4.

In a further preferred embodiment, CenH3 proteins as taught herein may comprise one active mutation that consists of:

a) an active mutation in the amino acid residue at position 9 of the amino acid sequence of SEQ ID NO: 12, or a variant thereof having at least 70%, more preferably at least 80%, even more preferably at least 90%, yet even more preferably at least 95%, most preferably at least 97%, 98% or 99% sequence identity to the amino acid sequence of SEQ ID NO: 12;

b) an active mutation in the amino acid residue at position 16 of the amino acid sequence of SEQ ID NO: 12, or a variant thereof having at least 70%, more preferably at least 80%, even more preferably at least 90%, yet even more preferably at least 95%, most preferably at least 97%, 98% or 99% sequence identity to the amino acid sequence of SEQ ID NO: 12; or

c) an active mutation in the amino acid residue at position 26 of the amino acid sequence of SEQ ID NO: 12, or a variant thereof having at least 70%, more preferably at least 80%, even more preferably at least 90%, yet even more preferably at least 95%, most preferably at least 97%, 98% or 99% sequence identity to the amino acid sequence of SEQ ID NO: 12. The protein of this embodiment is also denominated herein as an “Oryza sativa CenH3 mutant”.

Preferably, the active mutation at position 9 of the amino acid sequence of SEQ ID NO: 11 or SEQ ID NO: 12, or variants thereof as taught herein above, results in a modification into an amino acid residue selected from the group consisting of methionine, serine and threonine, more preferably into methionine.

Preferably, the active mutation at position 12 of the amino acid sequence of SEQ ID NO: 11 or at position 16 of the amino acid sequence of SEQ ID NO: 12, or variants thereof as taught herein above, results in a modification into an amino acid residue selected from the group consisting of methionine, serine and threonine, more preferably into serine.

Preferably, the active mutation at position 22 of the amino acid sequence of SEQ ID NO: 11 or at position 26 of the amino acid sequence of SEQ ID NO: 12, or variants thereof as taught herein above, results in a modification into an amino acid residue selected from the group consisting of glycine, alanine, valine, leucine and isoleucine, more preferably into leucine.

The CenH3 protein having an active specific mutation as taught herein may have the amino acid sequence as represented by SEQ ID NO: 13, SEQ ID NO: 14 or SEQ ID NO: 15.

The term ‘Oryza sativa CenH3_V9M amino acid sequence’ as used herein refers to a mutant Oryza sativa CenH3 protein amino acid sequence comprising a single point mutation in the amino acid residue at position 9 of SEQ ID NO: 13, which causes the modification of a valine to a methionine.

The term ‘Oryza sativa CenH3_P16S amino acid sequence’ as used herein refers to a mutant Oryza sativa CenH3 protein amino acid sequence comprising a single point mutation in the amino acid residue at position 16 of SEQ ID NO: 14, which causes the modification of a proline to a serine.

The term ‘Oryza sativa CenH3_P26L amino acid sequence’ as used herein refers to a mutant Oryza sativa CenH3 protein amino acid sequence comprising a single point mutation in the amino acid residue at position 26 of SEQ ID NO: 14, which causes the modification of a proline to a leucine.

Oryza sativa CenH3_V9M amino acid sequence, Oryza sativa CenH3_P16S amino acid sequence, Oryza sativa CenH3_P26L amino acid sequence are particularly advantageous for use as DH-inducer protein sequence, e.g. in plant breeding programs, for the generation of haploid progeny, or progeny with aberrant ploidy.

In an embodiment, Oryza sativa CenH3_V9M amino acid sequence (SEQ ID NO: 13), when present in a plant and when said plant is crossed with a wild-type plant, preferably a wild-type plant of the same species, at least 0.1, 0.5, 1 or 5% of the progeny generated is haploid or has aberrant ploidy. In an embodiment, Oryza sativa CenH3_P16S amino acid sequence (SEQ ID NO: 14), when present in a plant and when said plant is crossed with a wild-type plant, preferably a wild-type plant of the same species, at least 0.1, 0.5, 1 or 5% of the progeny generated is haploid or has aberrant ploidy. In an embodiment, Oryza sativa CenH3_P26L amino acid sequence (SEQ ID NO: 15), when present in a plant and when said plant is crossed with a wild-type plant, preferably a wild-type plant of the same species, at least 0.1, 0.5, 1 or 5% of the progeny generated is haploid or has aberrant ploidy.

In an embodiment, the CenH3 proteins or variants thereof, as taught herein, which is encoded by a CenH3 protein-encoding polynucleotide having one or more active specific mutation as taught above, which, when present in a plant in the absence of its endogenous CenH3-encoding polynucleotide and/or endogenous CenH3 protein, allows said plant to be viable, and allows generation of some haploid progeny, or progeny with aberrant ploidy, when said plant is crossed with a wild-type plant.

In an embodiment, any one of the CenH3 proteins or variants thereof as taught herein, may be derived from an endogenous CenH3 protein by introducing one or more active mutations in the polynucleotide encoding said endogenous CenH3 protein using targeted nucleotide exchange or by applying an endonuclease, e.g., in vitro. This may be particularly advantageous to generate a non-transgenic plant, for instance, by introducing in a plant cell (e.g., a protoplast) one or more active mutations in one or both alleles of the polynucleotide (CenH3 gene) encoding the endogenous CenH3 protein using targeted nucleotide exchange or by applying an endonuclease, and then grow the plant cells into plants.

In a preferred embodiment, the one or more active specific mutations in the CenH3 proteins or in the polynucleotides encoding CenH3 proteins and variants thereof, as taught herein, is or are point mutations, i.e. causing a change in a single amino acid at a specific position in the amino acid sequence or nucleic acid sequence.

In an embodiment, the CenH3 protein further comprises mutations in other sections of the protein, for instance in the C-terminal domain. Such further mutations are, for example, described by Karimi-Ashtiyani et al (2015) PNAS Vol: 112, pages 11211-11216 and Kuppu, et al. PLOS Genetics (2015) http://dx.doi.org/10.1371/journal.pgen.1005494, which are herein incorporated by reference.

CenH3-Encoding Polynucleotides Having an Active Mutation, Chimeric Gens, Vectors, and Host Cells

In a further aspect, the present invention relates to CenH3-encoding polynucleotides encoding any one of the CenH3 proteins and variants thereof as taught above.

Particularly, polynucleotides having nucleic acid sequences, such as cDNA, genomic DNA and RNA molecules, encoding any of the above CenH3 proteins or variants thereof are provided. Due to the degeneracy of the genetic code a variety of nucleic acid sequences may encode the same amino acid sequence. In the present invention, any polynucleotides capable of encoding CenH3 proteins or variants thereof as taught herein are referred to as ‘CenH3-encoding polynucleotides’. The polynucleotides provided include naturally occurring, artificial or synthetic nucleic acid sequences. It is understood that when sequences are depicted as DNA sequences while RNA is referred to, the actual base sequence of the RNA molecule is identical with the difference that thymine (T) is replace by uracil (U).

The present invention further relates to a polynucleotide encoding a CenH3 protein having an active mutation as taught herein. Said polynucleotide may be a synthetic, recombinant and/or isolated polynucleotide.

In an embodiment, the CenH3-encoding polynucleotide as taught herein may be a polynucleotide comprising the nucleic acid sequence of SEQ ID NO: 6 or SEQ ID NO: 9, or a variant thereof having at least 70%, more preferably at least 80%, even more preferably at least 90%, yet even more preferably at least 95%, most preferably at least 97%, 98% or 99% sequence identity to the nucleic acid sequence of SEQ ID NO: 6 or SEQ ID NO: 9, in which one or more nucleotides at positions 25-27 of the nucleic acid sequence of SEQ ID NO: 6 or at positions 25-27 of the nucleic acid sequence of SEQ ID NO: 9 are modified such that the polynucleotide encodes a plant CenH3 protein in which the amino acid sequence of SEQ ID NO: 2 has an altered residue at position 9 or 10, preferably at position 9 or SEQ ID NO: 3 has an altered residue at position 9.

In an embodiment, the residue at position 9 or 10 in the CenH3-encoding polynucleotides as taught above may be altered into any amino acid except a lysine, an arginine or a histidine. In a preferred embodiment, the residue at position 9 or 10 in the CenH3-encoding polynucleotides as taught above may be altered into an amino acid residue selected from the group consisting of serine, threonine, cysteine, tyrosine, glutamine, asparagine, glutamic acid and aspartic acid.

In a preferred embodiment, the residue at position 9 or 10 in the CenH3-encoding polynucleotides as taught above may be altered into an amino acid residue selected from a glutamic acid or an aspartic acid residue.

In an embodiment, the CenH3-encoding polynucleotide as taught herein may be a polynucleotide comprising the polynucleotide comprising the nucleic acid sequence of SEQ ID NO: 7 or SEQ ID NO: 10.

When present in a plant or a plant cell (e.g. protoplasm), the CenH3-encoding polynucleotides and variants thereof as taught herein (i.e. SEQ ID NO: 6, SEQ ID NO: 7, SEQ ID NO: 9 and SEQ ID NO: 10) comprising one or more active mutations or proteins encoded by said polynucleotides or variants thereof as taught therein, are capable of reducing or eliminating endogenous CenH3 activity to less than 90, 80, 70, 60, 50, 40, 30, 20, 10%, 5%, 4%, 3%, 2% or 1% of the CenH3 activity of the endogenous CenH3 protein in said plant or plant cell. CenH3 activity may be measured in vitro by measuring centromeric localization during separation of the chromosomes, for example, using a GFP fusion, where the level of fluorescence is a measure of CenH3 activity. Alternatively, yeast-2-hybrid interactions may be measured in vitro using all known proteins and/or centromeric DNA that interact with CenH3 protein. If the interaction is impaired, functionality of CenH3 is impaired.

In an embodiment, the present invention relates to a CenH3 polynucleotide comprising the nucleic acid sequence of SEQ ID NO: 6 or SEQ ID NO: 9, or a variant thereof having at least 70%, more preferably at least 80%, even more preferably at least 90%, yet even more preferably at least 95%, most preferably at least 97%, 98% or 99% sequence identity to the nucleic acid sequence of SEQ ID NO: 6 or SEQ ID NO: 9, but in which one or more nucleotides at positions 25-27 of the nucleic acid sequence of SEQ ID NO: 6 or at positions 25-27 of the nucleic acid sequence of SEQ ID NO: 9 are modified such that the polynucleotide encodes a plant CenH3 protein in which the amino acid sequence of SEQ ID NO: 2 or SEQ ID NO: 3 has an altered residue at position 9 or 10, preferably 9, as taught herein above, which is altered into any amino acid except for lysine, arginine or histidine. In a preferred embodiment, the altered residue is altered into an amino acid residue selected from the group consisting of serine, threonine, cysteine, tyrosine, glutamine, asparagine, glutamic acid and aspartic acid, and preferably into a glutamic acid or an aspartic acid residue.

In an embodiment, the Solanum lycopersicum CenH3 protein (SEQ ID NO: 3) as taught hereinabove may be encoded by a polynucleotide comprising the nucleic acid sequence of SEQ ID NO: 6 or SEQ ID NO: 9 in which one or more nucleotides at positions 25-27 of the nucleic acid sequence of SEQ ID NO: 6 or at positions 25-27 of the nucleic acid sequence of SEQ ID NO: 9 are mutated. In an embodiment, said mutation cause(s) a single amino acid change in the corresponding CenH3 protein at position 9 of SEQ ID NO: 3 or variants thereof as taught herein. The amino acid residue at position 9 may be substituted or changed for any amino acid except for lysine, arginine or histidine. In a preferred embodiment, the amino acid residue at position 9 is substituted by an amino acid selected from the group consisting of serine, threonine, cysteine, tyrosine, glutamine, asparagine, glutamic acid and aspartic acid, and preferably by a glutamic acid or an aspartic acid residue.

In an embodiment, the present invention relates to a CenH3 polynucleotide comprising the nucleic acid sequence of SEQ ID NO: 7 or SEQ ID NO: 10 and which encodes the Solanum lycopersicum CenH3_K9E protein (SEQ ID NO: 8) as taught herein.

In a further embodiment, the CenH3-encoding polynucleotide as taught herein may be a polynucleotide comprising the nucleic acid sequence of SEQ ID NO: 16 or SEQ ID NO: 20, or a variant thereof having at least 70%, more preferably at least 80%, even more preferably at least 90%, yet even more preferably at least 95%, most preferably at least 97%, 98% or 99% sequence identity to the nucleic acid sequence of SEQ ID NO: 16 or SEQ ID NO: 20, in which one or more nucleotides at positions 25-27, or 46-48 or 76-78, or a combination thereof, of the nucleic acid sequence of SEQ ID NO: 16 or at positions 25-27, or 46-48 or 76-78, or a combination thereof, of the nucleic acid sequence of SEQ ID NO: 20 are modified such that the polynucleotide encodes a plant CenH3 protein in which the amino acid sequence of SEQ ID NO: 11 has an altered residue at position 9, 12 and/or 22, preferably such that the polynucleotide encodes a plant CenH3 protein in which the amino acid sequence of SEQ ID NO: 12 has an altered residue at position at position 9, 16 or 26, or a combination thereof. Preferably, said altered residue at position 9 of the CenH3 encoding polynucleotides is altered into an amino acid residue selected from the group consisting of methionine, serine and threonine, more preferably into methionine. Preferably, said altered residue at position 12 or 16 of the CenH3 encoding polynucleotides is altered into an amino acid residue selected from the group consisting of methionine, serine and threonine, more preferably into serine. Preferably, said altered residue at position 22 or 26 of the CenH3 encoding polynucleotides is altered into an amino acid residue selected from the group consisting of glycine, alanine, valine, leucine and isoleucine, more preferably into leucine.

Preferably, the CenH3-encoding polynucleotide as taught herein relates to a CenH3 polynucleotide comprising the nucleic acid sequence of SEQ ID NO: 17 or SEQ ID NO: 21 and which encodes the Oryza sativa CenH3_V9M protein (SEQ ID NO: 13) as taught herein; or CenH3 polynucleotide comprising the nucleic acid sequence of SEQ ID NO: 18 or SEQ ID NO: 22 and which encodes the Oryza sativa CenH3_P16S protein (SEQ ID NO: 14) as taught herein; or CenH3 polynucleotide comprising the nucleic acid sequence of SEQ ID NO: 19 or SEQ ID NO: 23 and which encodes the Oryza sativa CenH3_P26L protein (SEQ ID NO: 15) as taught herein.

In an embodiment, the CenH3-encoding polynucleotides having an active mutation as taught herein are isolated. As used herein, the term ‘isolated CenH3-encoding polynucleotides’ refers to a nucleic acids which are substantially separated from other cellular components which naturally accompany a native plant sequence or protein, e.g. ribosomes, polymerases, many other plant genome sequences and proteins. The term embraces a nucleic acid sequence which has been removed from its naturally occurring environment and includes recombinant or cloned nucleic acid isolates and chemically synthesized analogs or analogs biologically synthesized by heterologous systems.’

In a further aspect, the present invention relates to a chimeric gene comprising any one of the CenH3-encoding polynucleotides as taught above.

In a further aspect, the present invention relates to a vector comprising any one of the CenH3-encoding polynucleotides as taught above or the chimeric gene as taught herein.

In a further aspect, the present invention relates to a host cell comprising any one of the CenH3-encoding polynucleotides as taught above or the chimeric gene as taught herein or the vector as taught herein. In an embodiment, the host cell is a plant cell, preferably a tomato plant cell, or a protoplast.

In an embodiment, the CenH3-encoding polynucleotides as taught above and variants thereof, as described above, may be particularly advantageous for making chimeric genes, and/or vectors for transfer of the CenH3 protein encoding polynucleotides into a host cell and production of the CenH3 protein(s) in host cells, such as cells, tissues, organs or organisms derived from transformed cell(s). Vectors for the production of CenH3 protein (or protein fragments or variants thereof) as taught herein in plant cells are herein referred to as ‘expression vectors’.

Suitable host cells for expression of CenH3 proteins include prokaryotes, yeast, or higher eukaryotic cells. Appropriate cloning and expression vectors for use with bacterial, fungal, yeast, and mammalian cellular hosts are described, for example, in Pouwels et al., Cloning vectors: A Laboratory Manual, Elsevier, N.Y., (1985). Cell-free translation systems could also be employed to produce the proteins of the present invention using RNAs derived from nucleic acid sequences disclosed herein.

Suitable prokaryotic host cells include gram-negative and gram-positive organisms, for example, Escherichia coli or Bacilli. Another suitable prokaryotic host cell is Agrobacterium, in particular Agrobacterium tumefaciens.

CenH3 proteins as taught herein can also be expressed in yeast host cells, for example from the Saccharomyces genus (e.g., Saccharomyces cerevisiae). Other yeast genera, such as Pichia or Kluyveromyces, can also be employed.

Alternatively, CenH3 proteins as taught herein may be expressed in higher eukaryotic host cells, including plant cells, fungal cells, insect cells, and mammalian, optionally non-human, cells.

In one embodiment, the present invention relates to a non-human organism modified to comprise a CenH3 polynucleotide as taught herein. The non-human organism and/or host cell may be modified by any methods known in the art for gene transfer including, for example, the use of delivery devices such as lipids and viral vectors, naked DNA, electroporation, chemical methods and particle-mediated gene transfer. In an advantageous embodiment, the non-human organism is a plant.

Any plant cell may be a suitable host cell. Suitable plant cells include those from monocotyledonous plants or dicotyledonous plants. For example, the plant may belong to the genus Solanum (including Lycopersicon), Nicotiana, Capsicum, Petunia and other genera. The following host species may suitably be used: Tobacco (Nicotiana species, e.g. N. benthamiana, N. plumbaginifolia, N. tabacum, etc.), vegetable species, such as tomato (L. esculentum, syn. Solanum lycopersicum) such as e.g. cherry tomato, var. cerasiforme or currant tomato, var. pimpinellifolium) or tree tomato (S. betaceum, syn. Cyphomandra betaceae), potato (Solanum tuberosum), eggplant (Solanum melongena), pepino (Solanum muricatum), cocona (Solanum sessiliflorum) and naranjilla (Solanum quitoense), peppers (Capsicum annuum, Capsicum frutescens, Capsicum baccatum), ornamental species (e.g. Petunia hybrida, Petunia axillaries, P. integrifolia), coffee (Coffea).

Alternatively, the plant may belong to any other family, such as to the Cucurbitaceae or Gramineae. Suitable host plants include for example maize/corn (Zea species), wheat (Triticum species), barley (e.g. Hordeum vulgare), oat (e.g. Avena sativa), sorghum (Sorghum bicolor), rye (Secale cereale), soybean (Glycine spp, e.g. G. max), cotton (Gossypium species, e.g. G. hirsutum, G. barbadense), Brassica spp. (e.g. B. napus, B. juncea, B. oleracea, B. rapa, etc), sunflower (Helianthus annus), safflower, yam, cassava, alfalfa (Medicago sativa), rice (Oryza species, e.g. O. sativa indica cultivar-group or japonica cultivar-group), forage grasses, pearl millet (Pennisetum spp. e.g. P. glaucum), tree species (Pinus, poplar, fir, plantain, etc), tea, coffea, oil palm, coconut, vegetable species, such as pea, zucchini, beans (e.g. Phaseolus species), cucumber, artichoke, asparagus, broccoli, garlic, leek, lettuce, onion, radish, turnip, Brussels sprouts, carrot, cauliflower, chicory, celery, spinach, endive, fennel, beet, fleshy fruit bearing plants (grapes, peaches, plums, strawberry, mango, apple, plum, cherry, apricot, banana, blackberry, blueberry, citrus, kiwi, figs, lemon, lime, nectarines, raspberry, watermelon, orange, grapefruit, etc.), ornamental species (e.g. Rose, Petunia, Chrysanthemum, Lily, Gerbera species), herbs (mint, parsley, basil, thyme, etc.), woody trees (e.g. species of Populus, Salix, Quercus, Eucalyptus), fibre species e.g. flax (Linum usitatissimum) and hemp (Cannabis sativa), or model organisms, such as Arabidopsis thaliana.

Preferred host cells are derived from ‘crop plants’ or ‘cultivated plants’, i.e. plant species which is cultivated and bred by humans. A crop plant may be cultivated for food or feed purposes (e.g. field crops), or for ornamental purposes (e.g. production of flowers for cutting, grasses for lawns, etc.). A crop plant as defined herein also includes plants from which non-food products are harvested, such as oil for fuel, plastic polymers, pharmaceutical products, cork, fibres (such as cotton) and the like.

In a preferred embodiment, the host cell is a cell from any plant. In a preferred embodiment, the host cell belongs to the Solanaceae family, more preferably to the genus Solanum, even more preferably to the species Solanum lycopersicum. Preferably, in this embodiment, the CenH3 polynucleotide comprised within the host cell is a Solanum lycopersicum CenH3 mutant as taught herein.

In a further preferred embodiment, the host cell is a monocotyledon, preferably belonging to the Poaceae family, more preferably to the genus Oryza, even more preferably to the species Oryza sativa. Preferably, in this embodiment, the CenH3 polynucleotide comprises within the host cell is a Oryza sativa CenH3 mutant as taught herein.

The construction of chimeric genes and vectors for, preferably stable, introduction of CenH3 protein-encoding nucleic acid sequences as taught herein into the genome of host cells is generally known in the art. To generate a chimeric gene the nucleic acid sequence encoding a CenH3 protein as taught herein is operably linked to a promoter sequence, suitable for expression in the host cells, using standard molecular biology techniques. The promoter sequence may already be present in a vector so that the CenH3 protein encoding nucleic acid sequence is simply inserted into the vector downstream of the promoter sequence. The vector may then be used to transform the host cells and the chimeric gene may be inserted in the nuclear genome or into the plastid, mitochondrial or chloroplast genome and expressed using a suitable promoter (e. g., Mc Bride et al., 1995 Bio/Technology 13, 362; U.S. Pat. No. 5,693,507). In an embodiment the chimeric gene as taught herein comprises a suitable promoter for expression in plant cells or microbial cells (e.g. bacteria), operably linked to a nucleic acid sequence encoding a CenH3 protein as taught herein, optionally followed by a 3′nontranslated nucleic acid sequence. The bacteria may subsequently be used for plant transformation (Agrobacterium-mediated plant transformation).

Plants Expressing CenH3 Polypeptides Having an Active Mutation

In a further aspect, the present invention relates to plants or plant cells expressing any one of the CenH3 polypeptides as taught herein or the chimeric gene as taught herein or the vector as taught herein.

In an embodiment, the plant or plant cell as taught herein may be any plant or plant cell. In a preferred embodiment, the plant or plant cell may belong to the family Solanaceae, more preferably to the genus Solanum, yet more preferably to the species Solanum lycopersicum. Preferably, said Solanum lycopersicum plant comprises a Solanum lycopersicum CenH3 mutant as taught herein.

In a further embodiment, the plant or plant cell as taught herein may be any plant or plant cell. In a preferred embodiment, the plant or plant cell is a monocotyledon that may belong to the family Poaceae, more preferably to the genus Oryza, yet more preferably to the species Oryza sativa, even more preferably of the subspecies Oryza sativa L. ssp. japonica. Preferably, said Oryza sativa L. ssp. japonica plant comprises an Oryza sativa CenH3 mutant as taught herein.

In an embodiment, the plants or plant cells as taught herein preferably do not express, or express at reduced levels (e.g., less than 90, 80, 70, 60, 50, 40, 30, 20, 10% of wild type levels), an endogenous CenH3 protein. For example, one can generate a mutation in an endogenous CenH3 protein that reduces or eliminates endogenous CenH3 protein activity or expression, or one can generate a knockout for endogenous CenH3 protein. In this case, one may generate a plant heterozygous for the gene knockout or mutation and introduce an expression vector for expression of a CenH3 protein having an active mutation as taught herein in the plant. Progeny from the heterozygote can then be selected that are homozygous for the mutation or knockout but that comprise the CenH3 protein having an active mutation.

Accordingly, in plants or plant cells as taught herein, preferably one or both endogenous CenH3 alleles are knocked out or mutated such that said plants or plant cells significantly or essentially completely lack endogenous CenH3 activity, i.e., sufficient to induce embryo lethality without complementary expression of a CenH3 protein having an active mutation as taught herein. In plants having more than a diploid set of chromosomes, all endogenous CenH3 alleles may be inactivated, mutated or knocked out. Alternatively, the expression of endogenous CenH3 protein may be silenced by any way known in the art, e.g. by introducing a siRNA or microRNA that reduces or eliminates expression of endogenous CenH3 protein. Ideally, the silencing agent is selected to silence the endogenous CenH3 protein but not the CenH3 protein having an active mutation.

In an embodiment, the plants or plant cells as taught herein may comprise one or two copies of an allele of a mutated polynucleotide encoding a CenH3 protein comprising an active mutation as taught herein. In an embodiment, the plants or plant cells as taught herein preferably comprise two copies of an allele of a mutated polynucleotide encoding a CenH3 protein comprising an active mutation as taught herein.

In an embodiment, any one of the polynucleotides as taught herein may be used for producing a haploid inducer line, e.g. for use in plant breeding.

CENH3 is a member of the kinetochore complex, the protein structure on chromosomes where spindle fibers attach during cell division. Without intending to limit the scope of the invention, it is believed that the observed results are at least partially due to generation of a kinetochore protein that acts more weakly than wildtype, thereby resulting in functional kinetochore complexes (for example, in mitosis), but which result in relatively poorly segregating chromosomes during meiosis relative to chromosomes also containing wildtype kinetochore complexes from the other parent. This results in functional kinetochore complexes when the altered protein is the only isoform in the cell, but relatively poorly segregating chromosomes during mitosis when the parent with altered kinetochores is crossed to a parent with wildtype kinetochore complexes. In addition to CENH3, other kinetochore proteins include, e.g., CENPC, MCM21, MIS 12, NDC80, and NUF2.

In one embodiment, the plants as taught herein may further express another recombinant mutated second kinetochore protein (including but not limited to CENPC, MCM21, MIS 12, NDC80, and NUF2) that disrupts the centromere, and/or plants in which at least one or both copies of an allele of CenH3 and of the endogenous second kinetochore protein gene has been knocked out, mutated to reduce or eliminate its function, or silenced. The present invention also provides for methods of generating a haploid plant by crossing a plant as taught herein and further expressing a mutated second kinetochore protein and not expressing an endogenous second kinetochore protein, to a plant that expresses an endogenous CenH3 protein and an endogenous second kinetochore protein.

Methods for the Generation of Plants

In a further aspect, the present invention relates to methods for making the plants or plant cells as taught herein above.

In an embodiment, the present invention relates to methods to modify an endogenous CenH3 gene using targeted mutagenesis methods (also referred to as targeted nucleotide exchange (TNE) or oligo-directed mutagenesis (ODM)). Targeted mutagenesis methods include, without limitation, those employing zinc finger nucleases, Cas9-like, Cas9/crRNA/tracrRNA or Cas9/gRNA CRISPR systems, or targeted mutagenesis methods employing mutagenic oligonucleotides, possibly containing chemically modified nucleotides for enhancing mutagenesis with sequence complementarity to the CenH3 gene, into plant protoplasts (e.g., KeyBase® or TALENs).

Alternatively, mutagenesis systems such as TILLING (Targeting Induced Local Lesions IN Genomics; McCallum et al., 2000, Nat Biotech 18:455, and McCallum et al. 2000, Plant Physiol. 123, 439-442, both incorporated herein by reference) may be used to generate plant lines which comprise a CenH3 gene encoding a CenH3 protein having an active mutation. TILLING uses traditional chemical mutagenesis (e.g. EMS mutagenesis) followed by high-throughput screening for mutations. Thus, plants, seeds and tissues comprising a CenH3 gene having the desired mutation may be obtained.

The methods as taught herein may comprise the steps of mutagenizing plant seeds (e.g. EMS mutagenesis), pooling of plant individuals or DNA, PCR amplification of a region of interest, heteroduplex formation and high-throughput detection, identification of the mutant plant, sequencing of the mutant PCR product. It is understood that other mutagenesis and selection methods may equally be used to generate such modified plants. Seeds may, for example, be radiated or chemically treated and the plants may be screened for a modified phenotype.

Modified plants may be distinguished from non-modified plants, i.e., wild type plants, by molecular methods, such as the mutation(s) present in the DNA, and by the modified phenotypic characteristics. The modified plants may be homozygous or heterozygous for the mutation.

In an embodiment, the present invention relates to a method for making a plant or plant cell as taught herein above, said method comprising the steps of: a) modifying a polynucleotide encoding an endogenous CenH3 protein within a plant cell to obtain a mutated polynucleotide encoding a CenH3 protein (i.e. CenH3-encoding polynucleotides having an active mutation as taught herein); b) selecting a plant cell comprising the mutated polynucleotide encoding a CenH3 protein; and c) optionally, regenerating a plant from said plant cell.

In an embodiment, the present invention relates to a method for making a plant as taught herein, which method comprises the steps of: a) modifying an endogenous plant CenH3 protein-encoding polynucleotide within a plant cell to obtain a plant CenH3 protein-encoding polynucleotide having one or more active mutations in its N-terminal tail domain; b) selecting a plant cell comprising the plant CenH3 protein-encoding polynucleotide having one or more active mutation; and c) optionally, regenerating a plant from said plant cell.

In an embodiment, the present invention relates to a method for making a plant as taught herein, comprising the steps of: a) transforming a plant cell with any one of the CenH3 polynucleotides as taught herein, or with a chimeric gene as taught herein, or with a vector as taught herein; b) selecting a plant cell comprising said CenH3 polynucleotide or chimeric gene or vector; and c) optionally, regenerating a plant from said plant cell.

In an embodiment, the methods for making a plant or plant cell as taught herein may further comprise the step of modifying an endogenous plant CenH3 protein-encoding polynucleotide or any other endogenous plant polynucleotide involved in expression of said polynucleotide within said plant cell to prevent expression of endogenous CenH3 protein.

In an embodiment, the CenH3 protein-encoding polynucleotides, preferably CenH3 protein-encoding chimeric gene, as taught herein can be stably inserted in a conventional manner into the nuclear genome of a single plant cell, and the so-transformed plant cell can be used in a conventional manner to produce a transformed plant that has an altered phenotype due to the presence of the CenH3 protein as taught herein in certain cells at a certain time. In this regard, a T-DNA vector, comprising a CenH3 protein-encoding polynucleotide as taught herein, in Agrobacterium tumefaciens can be used to transform the plant cell, and thereafter, a transformed plant can be regenerated from the transformed plant cell using the procedures described, for example, in EP 0 116 718, EP 0 270 822, PCT publication WO84/02913 and published European Patent application EP 0 242 246 and in Gould et al. (1991, Plant Physiol. 95,426-434). The construction of a T-DNA vector for Agrobacterium mediated plant transformation is well known in the art. The T-DNA vector may be either a binary vector as described in EP 0 120 561 and EP 0 120 515 or a co-integrate vector which can integrate into the Agrobacterium Ti-plasmid by homologous recombination, as described in EP 0 116 718.

Likewise, selection and regeneration of transformed plants from transformed plant cells is well known in the art. Obviously, for different species and even for different varieties or cultivars of a single species, protocols are specifically adapted for regenerating transformants at high frequency.

The resulting transformed plant can be used in a conventional plant breeding scheme to produce haploid plants that may subsequently become doubled haploid plants.

The invention also relates to a method of generating a haploid or doubled haploid plant, said method comprising the step of identifying a plant expressing an endogenous CenH3 protein and a plant as taught herein, wherein the plant as taught herein lacks expression of endogenous CenH3 protein at least in its reproductive parts and/or during embryonic development. The plant expressing an endogenous CenH3 protein may be crossed with a plant as taught herein, providing haploid plants.

In an embodiment, crossing does not comprise sexually crossing the whole genomes of plants. Instead, one set chromosomes is eliminated.

Methods for the Generation of Haploid Plants and/or Doubled Haploid Plants

In a further aspect, the present invention relates to a method of generating a haploid plant, a plant with aberrant ploidy or a doubled haploid plant, said method comprising the steps of: a) crossing a plant expressing an endogenous CenH3 protein to any one of the plants as taught herein; b) harvesting seeds; c) growing at least one seedling, plantlet or plant from said seeds; and d) selecting a haploid seedling, plantlet or plant or a seedling, a plantlet or a plant with aberrant ploidy, or a doubled seedling, plantlet or plant.

The skilled person is capable of selecting a haploid plant. Exemplary techniques include flow cytometry, or validation by specific SNP calling.

In an embodiment, the plant in step a) does not express an endogenous CenH3 protein at least in its reproductive parts and/or during embryonic development. In an embodiment, the plant expressing an endogenous CenH3 protein may be an F1 plant. The plant expressing an endogenous CenH3 protein may be a pollen parent of the cross, or may be an ovule parent of the cross.

Crossing a plant as taught herein, lacking expression of an endogenous CenH3 protein to take part in the kinetochore complex and expressing a CenH3 protein having an active mutation as taught herein, to a wild-type plant will result in at least some progeny that is haploid and comprises only chromosomes from the plant that expresses the endogenous CenH3 protein. Thus, the present invention allows for the generation of haploid plants having all of its chromosomes from a plant of interest by crossing the plant of interest with a plant expressing a CenH3 protein having an active mutation as taught herein, and collecting the resulting haploid seeds.

Thus, genome elimination can be engineered with a precise molecular change independent of parental genotype. CenH3 protein is found in any plant species. This allows haploid plants to be made in species where conventional methods for haploid plant production, such as tissue culture of haploid cells and wide crosses, are unsuccessful.

The plant expressing a CenH3 protein having an active mutation as taught herein may be crossed as either the male or female parent. The methods as taught herein allow for transfer of paternal chromosomes into maternal cytoplasm. Thus, it can generate cytoplasmic male sterile lines with a desired genotype in a single step.

In a further aspect, the present invention relates to a method of generating a doubled haploid plant, said method comprising the steps of: converting the haploid seedling, plantlet or plant obtained in step d) as taught herein into a doubled haploid plant.

In an embodiment, the converting of the haploid seedling, plantlet or plant into a doubled haploid plant may be performed using colchicine.

In a further aspect, the present invention relates to a method of generating a doubled haploid plant, said method comprising the steps of: a) crossing a plant expressing an endogenous CenH3 protein with any one of the modified plant as taught herein; selecting a haploid plant; and converting said haploid plant into a doubled haploid plant.

In an embodiment, the crossing step a) is performed at a temperature in the range of about 24° C. to about 30° C.

Thus, once generated, haploid plants can be used for the generation of doubled haploid plants, which comprise an exact duplicate copy of chromosomes. A wide variety of methods are known for generating doubled haploid organisms from haploid organisms. For example, chemicals such as colchicine may be applied to convert the haploid plant into a doubled haploid plant. Alternatively, ploidy may double spontaneously during embryonal development or at a later developmental stage of a plant.

In an embodiment, the methods for generation of haploid plants, plants with aberrant ploidy and/or doubled haploid plants as taught herein do not comprise sexually crossing the whole genomes of said plant. Instead, one set of chromosomes is eliminated during the cross.

Doubled haploid plants can be further crossed to other plants to generate F1, F2, or subsequent generations of plants with desired traits.

Doubled haploids plants may be obtained that do not bear transgenic or mutagenized genes. Additionally, doubled haploid plants can rapidly create homozygous F2s from a hybrid F1.

In an embodiment, the plant expressing an endogenous CenH3 protein may be a pollen parent of the cross. In a further embodiment, the plant expressing an endogenous CenH3 protein may be an ovule parent of the cross.

Solanum lycopersicum Plant or Seeds

In a further aspect, the present invention relates to a Solanum lycopersicum plant or seed comprising the CenH3 protein sequence of SEQ ID NO: 3 and further comprising one or more active mutations in its N-terminal tail domain. Such plant may comprise the Solanum lycopersicum CenH3 mutant as taught herein.

In an embodiment, the one or more active mutations are in the plant CenH3 motif block 1 (SEQ ID NO: 4).

In a preferred embodiment, the one or more active mutations are in the consensus Solanaceae CenH3 motif block 1 (SEQ ID NO: 5) as taught herein.

In an embodiment, the active mutation is in the amino acid residue at position 9 of SEQ ID NO: 4 or SEQ ID NO: 5, or variants thereof as defined herein, said amino acid residue being modified into any amino acid except a lysine, an arginine or a histidine.

In a preferred embodiment, the amino acid residue may be modified by another amino acid residue selected from the group consisting of serine, threonine, cysteine, tyrosine, glutamine, asparagine, glutamic acid and aspartic acid.

In a more preferred embodiment, the amino acid residue may be modified by another amino acid residue selected from a glutamic acid or an aspartic acid residue.

In an embodiment, the Solanum lycopersicum plant or seed as taught herein may comprise any one of the polynucleotide as taught herein or the chimeric gene as taught herein, or the vector as taught herein.

In a preferred embodiment, the Solanum lycopersicum plant or seed as taught herein may comprise a polynucleotide encoding a protein comprising the amino acid sequence of SEQ ID NO: 8.

In a further preferred embodiment, the Solanum lycopersicum plant or seed as taught herein may comprise a polynucleotide comprising the nucleic acid sequence of SEQ ID NO: 7 or SEQ ID NO: 10.

In an embodiment, the Solanum lycopersicum plant or seed as taught herein may comprise a polynucleotide that encodes a CenH3 protein as taught herein.

In an embodiment, the Solanum lycopersicum plants or seeds as taught herein do not express an endogenous CenH3 protein or do not have a functional endogenous CenH3 protein, at least in the reproductive parts and/or during embryonic development.

The Solanum lycopersicum plant or seed as taught herein, wherein the endogenous CenH3 protein is not expressed at least in the reproductive parts and/or during embryonic development.

In an embodiment, the Solanum lycopersicum plant or cells as taught herein may be used for producing a haploid Solanum lycopersicum plant.

In a further embodiment, the Solanum lycopersicum plant or cell as taught herein may be used for producing a doubled haploid Solanum lycopersicum plant.

Oryza sativa Plant or Seeds

In a further aspect, the present invention relates to a Oryza sativa plant or seed, preferably to a Oryza sativa L. ssp. Japonica, comprising the CenH3 protein sequence of SEQ ID NO: 12 and further comprising one or more active mutations in its N-terminal tail domain. Such plant may comprise the Oryza sativa CenH3 mutant as taught herein.

In an embodiment, the one or more active mutations are in the plant CenH3 motif block 1 (SEQ ID NO: 4). The active mutation may be in the amino acid residue at position 9 of SEQ ID NO: 4, or at the amino acid residue at position 9 or SEQ ID NO: 12 or variants thereof as defined herein, said amino acid residue being modified into any amino acid except a valine. The amino acid residue may be modified by another amino acid residue selected from the group consisting of methionine, serine or threonine, preferably a methionine.

In a further embodiment, the one or more active mutations are in the plant CenH3 N-terminal tail, preferably at position 16 of SEQ ID NO: 12 or variants thereof as defined herein, said amino acid residue being modified into any amino acid except a proline. The amino acid residue may be modified by another amino acid residue selected from the group consisting of methionine, serine and threonine, more preferably into serine.

In a further embodiment, the one or more active mutations are in the plant CenH3 N-terminal tail, preferably at position 26 of SEQ ID NO: 12 or variants thereof as defined herein, said amino acid residue being modified into any amino acid except a proline. The amino acid residue may be modified by another amino acid residue selected from the group consisting of glycine, alanine, valine, leucine and isoleucine, more preferably into leucine.

In an embodiment, the Oryza sativa plant or seed as taught herein may comprise any one of the polynucleotide as taught herein or the chimeric gene as taught herein, or the vector as taught herein.

In a preferred embodiment, the Oryza sativa plant or seed as taught herein may comprise a polynucleotide encoding a protein comprising the amino acid sequence of SEQ ID NO: 13, SEQ ID NO: 14 or SEQ ID NO: 15.

In a further preferred embodiment, the Oryza sativa plant or seed as taught herein may comprise a polynucleotide comprising the nucleic acid sequence of SEQ ID NO: 17, SEQ ID NO: 18 or SEQ ID NO: 19 or SEQ ID NO: 21, SEQ ID NO: 22 or SEQ ID NO: 23.

In an embodiment, the Oryza sativa plant or seed as taught herein may comprise a polynucleotide that encodes a CenH3 protein as taught herein.

In an embodiment, the Oryza sativa plants or seeds as taught herein do not express an endogenous CenH3 protein or do not have a functional endogenous CenH3 protein, at least in the reproductive parts and/or during embryonic development.

The Oryza sativa plant or seed as taught herein, wherein the endogenous CenH3 protein is not expressed at least in the reproductive parts and/or during embryonic development.

In an embodiment, the Oryza sativa plant or cells as taught herein may be used for producing a haploid Oryza sativa plant.

In a further embodiment, the Oryza sativa plant or cell as taught herein may be used for producing a doubled haploid Oryza sativa plant.

Uses

In a further aspect, the present invention relates to uses of the CenH3 proteins comprising one or more active mutations and/or CenH3-encoding polynucleotides having one or more active mutation as well as chimeric genes, vectors and host cells comprising them for producing a haploid inducer line or plant, e.g. for use in plant breeding.

In a further aspect, the present invention relates to the Solanum lycopersicum plant, plantlet or seeds as taught herein for producing a haploid Solanum lycopersicum plant and/or for producing a doubled haploid Solanum lycopersicum plant.

In a further aspect, the present invention relates to the Oryza sativa plant, plantlet or seeds as taught herein for producing a haploid Oryza sativa plant and/or for producing a doubled haploid Oryza sativa plant. Preferably, the present invention relates to the Oryza sativa L. ssp. Japonica plant, plantlet or seeds as taught herein for producing a haploid Oryza sativa L. ssp. Japonica plant and/or for producing a doubled haploid Oryza sativa L. ssp. Japonica plant.

### BRIEF DESCRIPTION OF THE FIGURES

FIG. 1. depicts a micronucleus (arrow) in pollen tetrad of CenH3_K9E, DAPI staining.

### SEQUENCE LISTING

SEQ ID NO: 1: Plant CenH3 consensus protein sequence

SEQ ID NO: 2: Consensus Solanaceae CenH3 protein sequence

SEQ ID NO: 3: Solanum lycopersicum CenH3 protein sequence (Solyc01g095650.2.1)

SEQ ID NO: 4: Plant consensus CenH3 motif 1 domain protein sequence

SEQ ID NO: 5: Consensus Solanaceae CenH3 motif 1 domain protein sequence

SEQ ID NO: 6: Solanum lycopersicum CenH3 coding sequence (Solyc01g095650.2.1)

SEQ ID NO: 7: Solanum lycopersicum CenH3_K9E coding sequence

SEQ ID NO: 8: Solanum lycopersicum CenH3_K9E protein sequence

SEQ ID NO: 9: Solanum lycopersicum CenH3 genomic DNA sequence (Solyc01g095650.2.1)

SEQ ID NO: 10: Solanum lycopersicum CenH3_K9E genomic DNA sequence

SEQ ID NO: 11: Monocotyledon consensus CenH3 protein sequence

SEQ ID NO: 12: Oryza sativa L. ssp. japonica CenH3 protein sequence (LOC_Os05g41080)

SEQ ID NO: 13: Oryza sativa L. ssp. japonica CenH3_V9M protein sequence

SEQ ID NO: 14: Oryza sativa L. ssp. japonica CenH3_P16S protein sequence

SEQ ID NO: 15: Oryza sativa L. ssp. japonica CenH3_P26L protein sequence

SEQ ID NO: 16: Oryza sativa L. ssp. japonica CenH3 coding sequence (LOC_Os05g41080)

SEQ ID NO: 17: Oryza sativa L. ssp. japonica CenH3_V9M coding sequence

SEQ ID NO: 18: Oryza sativa L. ssp. japonica CenH3_P16S coding sequence

SEQ ID NO: 19: Oryza sativa L. ssp. japonica CenH3_P26L coding sequence

SEQ ID NO: 20: Oryza sativa L. ssp. japonica CenH3 genomic DNA sequence (LOC_Os05g41080)

SEQ ID NO: 21: Oryza sativa L. ssp. japonica CenH3_V9M genomic DNA sequence

SEQ ID NO: 22: Oryza sativa L. ssp. japonica CenH3_P16S genomic DNA sequence

SEQ ID NO: 23: Oryza sativa L. ssp. japonica CenH3_P26L genomic DNA sequence

### EXAMPLES

### Example 1: Generation of a Haploid Plant

Plant Material

Three tomato cultivars were used namely ‘MoneyBerg TMV+’, ‘MicroTom’ and ‘RZ52201’. From a tomato RZ52201 mutant population, following methods described in WO 2007/037678 and WO2009/041810, a somatic non-synonymous mutant in the gene CenH3 was selected, namely CenH3_K9E, which is mutated at amino acid position 9. The selected mutant plant was self-pollinated and in the offspring, plants were selected that were homozygous for the mutated locus. From a tomato MoneyBerg TMV+ mutant population a somatic synonymous mutant was selected, following methods described in WO 2007/037678 and WO2009/041810, in the gene Msi2, namely Msi2_D337D, which is mutated at amino acid position 337 (C to T). The selected mutant plant was self-pollinated and in the offspring, plants were selected that were homozygous for the mutated locus.

Method

Uniparental genome elimination and the resulting production of a haploid plant was provoked by making a cross between a so-called haploid inducer line and another non-haploid inducer line, for example a breeding line. Crosses of tomato lines for uniparental genome elimination were performed at relatively high temperatures (26-28° C.), since it is known that an elevated temperature can, but only in some cases, have a positive effect on the occurrence of uniparental genome elimination (Sanei et al. PNAS 108.33 (2011): E498-E505).

Results

The non-synonymous mutation of A to G in the CenH3_K9E mutant resulted in an amino acid modification of a lysine to a glutamate (SEQ ID NO: 8). The synonymous mutation of C to T in the Msi2_D337D mutant did not result in an amino acid modification. Both mutant plants homozygous for the CenH3_K9E or the Msi2_D337D mutation were used as pollen donor and as female in crosses at relatively high temperatures (26-28° C.) using non-mutated wild type MicroTom plants as female or pollen donor, respectively. Table 1 lists an overview of all crosses made and the sown seeds which were evaluated for the MicroTom phenotype.

TABLE 1
List of crosses made; genetic background of the parents used,
number of offspring plants tested and number of offspring plants
which showed MicroTom dwarf phenotype. Experiments with
MicroTom as female are shown from two subsequent years.

| | | | Number | Number of | Year |
|---|---|---|---|---|---|
| | | | of | plants with | cross |
| Plant used as | Plant used as | Background | plants | MicroTom | was |
| female | male | mutant parent | tested | phenotype | made |
| MicroTom | CenH3_K9E | RZ52201 | 516 | 6 | 2014 |
| CenH3_K9E | MicroTom | RZ52201 | 564 | 1 | 2015 |
| MicroTom | CenH3_K9E | RZ52201 | 297 | 13 | 2015 |
| RZ52201 | MicroTom | — | 188 | 0 | 2015 |
| MicroTom | RZ52201 | — | 188 | 0 | 2015 |
| MoneyBergTMV+ | MicroTom | — | 188 | 0 | 2015 |
| MicroTom | MoneyBergTMV+ | — | 188 | 0 | 2015 |
| Msi2_D337D | MicroTom | MoneyBergTMV+ | 160 | 0 | 2015 |
| MicroTom | Msi2_D337D | MoneyBergTMV+ | 36 | 0 | 2015 |

Seeds derived from the crosses listed in table 1 were sown and the plants were evaluated for their DNA content by means of flow cytometry. The flow cytometry analysis resulted in a determination of only normal diploid ploidy levels for all plants tested, similar to wild type tomato cultivars such as MoneyBergTMV+. A single exception was found; for the cross in 2014 of MicroTom (female)×CenH3_K9E (male), one offspring plant was found to be aneuploid (i.e., having an aberrant ploidy) based on flow cytometry analysis.

The cultivar MicroTom has a dwarf phenotype, which is known to be recessive (Marti et al, J Exp Bot, Vol. 57, No. 9, pp. 2037-2047, 2006). After a cross of MicroTom to or with, for instance a MoneyBerg TMV+ or RZ52201 wild type cultivar, one only finds offspring with the indeterminate non-dwarf phenotype of the MoneyBerg TMV+ or RZ52201 wild type cultivar. The same was found for crosses with the Msi2_D337D synonymous mutant and MicroTom; all offspring of a MicroTom and Msi2_D337D mutant crosses showed the indeterminate non-dwarf phenotype of the MoneyBerg TMV+ parent. Using the CenH3_K9E mutant as male or female parent, in total 20 plants were found which showed a MicroTom phenotype. This indicates that the RZ52201 parent genetic material is not part of the resulting offspring and this indicates that these 20 offspring plants are of haploid MicroTom origin. The ploidy of all plants of the latter 20 plants was found to be diploid, indicating that spontaneous doubling had occurred, a phenomena which has been described to have an exceptional high frequency of appearance for tomato (Report of the Tomato Genetics Cooperative Number 62—December 2012).

In order to determine whether and to what extent uniparental genome elimination had occurred, a single nucleotide polymorphism (SNP) assay was run for in total 24 positions, 2 SNPs on each of the 12 tomato chromosomes for the 2015 crosses. For the 2014 crosses SNP assays were run on in total 8 positions, one on chromosomes 2, 7, 9, and 12 and two on chromosomes 3 and 6. The single nucleotide polymorphisms selected were homozygous for one base pair for the MicroTom parent and homozygous for all but not the MicroTom base pair in the RZ52201 parent. A regular cross between a wild type MicroTom cultivar and the RZ52201 cultivar would result in a heterozygous single nucleotide polymorphism score.

However, when the process of uniparental genome elimination has occurred, one expects the loss of the haploid inducer line genome. The single nucleotide polymorphism test resulted in calling of only homozygous base pair scores from the MicroTom parent for each of the 20 offspring plants which also showed the MicroTom phenotype and none of the RZ52201 parent were called. Based on the single nucleotide polymorphism scores it was concluded that the complete genome of the CenH3_K9E mutant was no longer present in the offspring.

Therefore, it can be concluded that the CenH3_K9E mutant functions as a highly efficient haploid inducer line. In the crosses in which the CenH3_K9E mutant was used as female parent, a selfing of MicroTom can be ruled out. It is highly unlikely that in the experiment using MicroTom as female parent selfing took place, given the very low number of offspring showing the MicroTom phenotype in two subsequent years of making crosses (only 6 seeds out of 516 and 13 out of 297), and the fact that only homozygous base pairs were scored.

Pollen tetrads of the CenH3_K9E mutant and of RZ52201 control plants were checked for occurrence of aberrancies.

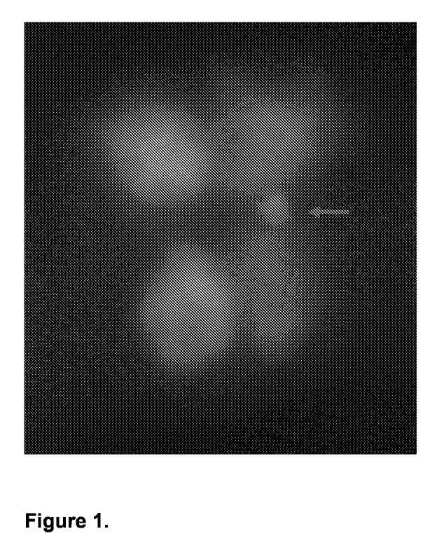

From two flowers, the anthers were squashed in order to look at pollen tetrads. For the CenH3_K9E mutant, scoring 2 flowers an average 2.60±0.25 percent of micronuclei were observed in all tetrads. FIG. 1 shows an example of such a micronucleus. For the RZ52201 control, rarely an anther was observed containing pollen tetrads with micronuclei. Scoring 5 flowers an average 0.58±0.36 percent of micronuclei were observed in all tetrads. It is concluded that the separation of chromosomes during meiosis is considerably more frequently disturbed as a result on the CenH3_K9E mutation compared to the control. Aberrant mitosis, for instance observations of micronuclei, are often used as direct evidences of chromosome elimination and haploid production in inter-, intra-specific hybridizations in crops. For example, aberrant mitosis as well as aberrant meiosis, for instance micronuclei, were found in a study of a maize DH-inducer line (Qiu, Fazhan, et al. Current Plant Biology 1 (2014): 83-90). The observations of meiosis micronuclei in the CenH_K9E mutant, suggest that during mitosis similar processes occur. It is likely that the process of uniparental genome elimination during the first mitotic divisions after fusion of wild type and CenH_K9E zygotes takes place and that this results in the observed induction of haploids.

### Example 2: Uniparental Genome Elimination in Rice

Plant Material

Oryza sativa L. ssp. japonica cv. Volano are used to generate a mutant population by means of chemical mutagenesis. From this mutant population, following methods described in WO2007/037678 and WO2009/041810, three somatic non-synonymous mutants in the gene CenH3 (LOC_Os05g41080; SEQ ID NO: 13, SEQ ID NO: 14 and SEQ ID NO: 15) are selected, namely CenH3_V9M, CenH3_P16S and CenH3_P26L. The selected mutant plants are self-pollinated and in the offspring, plants are selected that are homozygous for the mutated locus. A non-mutated Oryza sativa L. ssp. japonica (encoding SEQ ID NO: 12) cv. Volano plant is used as well.

Method

Uni-parental genome elimination and the resulting production of a haploid plant is provoked by making a cross between a so called haploid inducer line and another non-haploid inducer line, for example a non-mutated Oryza sativa L. ssp. japonica cv. Volano plant. The ploidy of the offspring is measured to determine whether they are diploid or haploid. To include the possibility that a haploid offspring plant is spontaneously doubled to a diploid state, the total absence of either of the three listed CenH3 mutant SNPs is tested as well. In a spontaneously doubled provoked haploid plant none of three separate CenH3 mutant SNPs (SEQ ID NO: 13, SEQ ID NO: 14 or SEQ ID NO: 15), not even as heterozygous allele, will be present.

Results

The non-synonymous mutation of G to A in the CenH3_V9M mutant resulted in an amino acid modification of a valine to a methionine (SEQ ID NO: 13). The non-synonymous mutation of C to T in the CenH3_P16S mutant resulted in an amino acid modification of a proline to a serine (SEQ ID NO: 14). The non-synonymous mutation of C to T in the CenH3_P26L mutant resulted in an amino acid modification of a proline a leucine (SEQ ID NO: 15). Each of the three mutant plants homozygous for the CenH3_V9M, the CenH3_P16S or the CenH3_P26L mutation were used as pollen donor using non-mutated wild type Oryza sativa L. ssp. japonica cv. Volano as female. Table 2 lists an overview of all crosses and the seeds that are sown which are evaluated for ploidy levels. A reciprocal cross may yield similar results.

TABLE 2
Example list of crosses which can be made; genetic background
of all plants is Oryza sativa L. ssp. japonica cv. Nipponbare,
number of offspring plants which are tested and number of
haploid offspring plants based on flow cytometry.

| Plant used as | Plant used | Number of plants | Number of haploid |
|---|---|---|---|
| female | as male | tested | plants |
| Wild type | CenH3_V9M | 300 | 3 |
| Wild type | CenH3_P16S | 300 | 1 |
| Wild type | CenH3_P26L | 300 | 2 |
| Wild type | Wild type | 300 | 0 |

Seeds derived from the crosses listed in table 1 are sown and the plants are evaluated for their DNA content by means of flow cytometry. Presence of CenH3_V9M, the CenH3_P16S or CenH3_P26L mutant SNP is tested in plants determined to be haploid by flow cytometry analysis. Absence of the mutant SNP indicates that the mutant parent genetic material is not part of the resulting offspring and that these offspring plants are of haploid wild type parent origin, i.e. that each of the CenH3_V9M, the CenH3_P16S or the CenH3_P26L mutants function as a highly efficient haploid inducer line.

1-60. (canceled)

61. A CenH3 protein of plant origin comprising one or more active mutations in the CenH3 motif block 1 of the N-terminal tail domain having an amino acid sequence of SEQ ID NO: 4.

62. The CenH3 protein according to claim 61, wherein the active mutation is at position 9 or 10 of SEQ ID NO: 4.

63. The CenH3 protein according to claim 61, which is derived from an endogenous CenH3 protein having at least 70% sequence identity to any one of SEQ ID NO: 1, 2, 3, 11 and 12, or is encoded by a polynucleotide having at least 70% sequence identity to any one of SEQ ID NO: 6, 9, 16, or 20.

64. The CenH3 protein according to claim 63, wherein the derivation is by introducing mutations in the polynucleotide encoding the endogenous CenH3 protein using targeted nucleotide exchange or by applying an endonuclease.

65. The CenH3 protein according to claim 61, wherein the active mutation is a point mutation.

66. The CenH3 protein according to claim 61, wherein the CenH3 protein of plant origin comprises the amino acid sequence of SEQ ID NO: 8 or 13, or is encoded by a polynucleotide comprising the nucleic acid sequence of SEQ ID NO: 7, 10, 17 or 21.

67. A polynucleotide encoding the CenH3 protein according to claim 61.

68. The polynucleotide according to claim 67, comprising the nucleic acid sequence of SEQ ID NO: 7, 10, 17 or 21.

69. A chimeric gene comprising the polynucleotide according to claim 67.

70. A vector comprising the polynucleotide according to claim 67.

71. A vector comprising the chimeric gene according to claim 68.

72. A host cell comprising the polynucleotide according to claim 67.

73. The host cell according to claim 72, wherein the host cell is a plant cell.

74. The host cell according to claim 73, wherein the plant cell is a tomato or rice plant cell or a tomato or rice protoplast.

75. A plant expressing the CenH3 protein according to claim 61.

76. The plant according to claim 75, wherein endogenous CenH3 protein is not expressed.

77. The plant according to claim 76, wherein the plant is a Solanum plant or an Oryza plant.

78. The plant according to claim 77, wherein the plant is a Solanum lycopersicum plant or an Oryza sativa plant.

79. A method for making a plant according to claim 75, comprising the steps of:
(a) modifying a polynucleotide encoding an endogenous CenH3 protein within a plant cell to obtain a mutated polynucleotide encoding a CenH3 protein of plant origin comprising one or more active mutations in the CenH3 motif block 1 of the N-terminal tail domain having an amino acid sequence of SEQ ID NO: 4;
(b) selecting a plant cell comprising the mutated polynucleotide; and
(c) optionally, regenerating a plant from the plant cell.

80. A method of generating a haploid plant, a plant with aberrant ploidy or a doubled haploid plant, the method comprising the steps of:
(a) crossing a plant expressing an endogenous CenH3 protein to the plant of claim 75, wherein the plant according to claim 75 does not express an endogenous CenH3 protein at least in its reproductive parts and/or during embryonic development;
(b) harvesting seed;
(c) growing at least one seedling, plantlet or plant from the seed; and
(d) selecting a haploid seedling, plantlet or plant; a seedling, plantlet or plant with aberrant ploidy; or a doubled haploid seedling, plantlet or plant.

81. A method of generating a doubled haploid plant, the method comprising the step of:
(a) crossing a plant expressing an endogenous CenH3 protein to the plant of claim 75, wherein the plant according to claim 75 does not express an endogenous CenH3 protein at least in its reproductive parts and/or during embryonic development;
(b) harvesting seed;
(c) growing at least one seedling, plantlet or plant from the seed,
(d) selecting a haploid seedling, plantlet or plant; a seedling, plantlet or plant with aberrant ploidy; or a doubled haploid seedling, plantlet or plant; and
(e) converting the haploid seedling, plantlet or plant into a doubled haploid plant.


SEQUENCE LISTING (23 sequences)

SEQ ID NO: 1 - protein, 149 aa, Artificial Sequence
  Description of Artificial Sequence Synthetic Plant CenH3 consensus sequence
  MOD_RES (9)..(9): Any naturally occurring amino acid
  MOD_RES (35)..(35): Any naturally occurring amino acid
MARTKHFAXRSRRTKAASSQAAGPSTPRGAQTTPXAKRARQAPGSQKKPHRYRPGTVALREIRKFQKSTNLLIPAAPFIRLVREITNALAPEVTRWTAEALVALQEAAEDYLVGLFEDAMLCAIHAKRVTLMRKDFELARRLGGKGRPW

SEQ ID NO: 2 - protein, 148 aa, Artificial Sequence
  Description of Artificial Sequence Synthetic Consensus Solanaceae CenH3 protein sequence
  MOD_RES (14)..(14): Any naturally occurring amino acid
  MOD_RES (17)..(17): Any naturally occurring amino acid
  MOD_RES (21)..(21): Any naturally occurring amino acid
  MOD_RES (24)..(24): Any naturally occurring amino acid
  MOD_RES (32)..(32): Any naturally occurring amino acid
  MOD_RES (34)..(34): Any naturally occurring amino acid
  MOD_RES (41)..(41): Any naturally occurring amino acid
  MOD_RES (49)..(49): Any naturally occurring amino acid
  MOD_RES (63)..(63): Any naturally occurring amino acid
  MOD_RES (91)..(91): Any naturally occurring amino acid
  MOD_RES (118)..(118): Any naturally occurring amino acid
  MOD_RES (145)..(145): Any naturally occurring amino acid
MARTKHLARKSRTXPSXAAGXSAXPQSTPTRXSXRSAPATXGVQKPKKXRYRPGTVALREIRXFQKTWNLLIPAAPFIRLVREISHFFAPXVTRWQAEALIALQEAAEDFLVHLFEDXMLCAIHAKRVTLMKKDFELARRLGGKXRPW

SEQ ID NO: 3 - protein, 144 aa, Solanum lycopersicum
MARTKHLAKRSRTTSAAPSATPSTPSRKSPRSAPATSVQKPKQKKRYRPGTVALREIRHFQKTWDLLIPAAPFIRLVREISHFYAPGVTRWQAEALIAIQEAAEDFLVHLFEDAMLCAIHAKRVTLMKKDFELARRLGGKGQPW

SEQ ID NO: 4 - protein, 11 aa, Artificial Sequence
  Description of Artificial Sequence Synthetic Plant CenH3 motif 1 domain consensus sequence
  MOD_RES (7)..(7): Phe or Thr
  MOD_RES (9)..(9): Any amino acid or may be absent
  MOD_RES (10)..(10): Arg or Lys
  MOD_RES (11)..(11): Lys or Ser
MARTKHXAXXX

SEQ ID NO: 5 - protein, 11 aa, Artificial Sequence
  Description of Artificial Sequence Synthetic Consensus Solanaceae CenH3 motif 1 domain protein sequence
  MOD_RES (6)..(6): His or Gln
  MOD_RES (7)..(7): Met, Leu, or Thr
  MOD_RES (9)..(10): Arg or Lys
MARTKXXAXXS

SEQ ID NO: 6 - DNA, 435 bp, Solanum lycopersicum
atggcgagaaccaaacacctcgcgaaacgaagtcgcaccacttctgctgcaccttcagcgactccatcgacgccttcaagaaaaagtccaaggtctgcaccggcaacttcagtgcagaagccaaaacaaaagaagcgttacaggccagggacagtggcacttcgagaaatcagacactttcagaagacgtgggatcttcttattccagctgctcctttcatcagacttgttagagaaattagtcacttttatgcacctggggtaactcgttggcaagctgaggcgttaattgctattcaagaggctgctgaagattttttagttcatttgtttgaagatgcaatgctatgtgctattcatgcgaagcgtgttacacttatgaaaaaagattttgagctggctcgacgacttggaggaaaaggacaaccttggtga

SEQ ID NO: 7 - DNA, 435 bp, Solanum lycopersicum
atggcgagaaccaaacacctcgcggaacgaagtcgcaccacttctgctgcaccttcagcgactccatcgacgccttcaagaaaaagtccaaggtctgcaccggcaacttcagtgcagaagccaaaacaaaagaagcgttacaggccagggacagtggcacttcgagaaatcagacactttcagaagacgtgggatcttcttattccagctgctcctttcatcagacttgttagagaaattagtcacttttatgcacctggggtaactcgttggcaagctgaggcgttaattgctattcaagaggctgctgaagattttttagttcatttgtttgaagatgcaatgctatgtgctattcatgcgaagcgtgttacacttatgaaaaaagattttgagctggctcgacgacttggaggaaaaggacaaccttggtga

SEQ ID NO: 8 - protein, 144 aa, Solanum lycopersicum
MARTKHLAERSRTTSAAPSATPSTPSRKSPRSAPATSVQKPKQKKRYRPGTVALREIRHFQKTWDLLIPAAPFIRLVREISHFYAPGVTRWQAEALIAIQEAAEDFLVHLFEDAMLCAIHAKRVTLMKKDFELARRLGGKGQPW

SEQ ID NO: 9 - DNA, 5193 bp, Solanum lycopersicum
atggcgagaaccaaacacctcgcgaaacgaagtcgcaccacttctggtacttctctacctctcctttttataatttaacctaactgcacacatatcgtaattttagagttttgaaaaagattagcgaattcgcaaatacaagtaagaagatctgacttagtttttaatacatatttgtaaagaaaaaaaatcaactgtatatggctcaagttgaagataagaagcggtgattaatacctttaatgtaaattcagcgatggtttctctgtgtgtgtgtgaatgtttacattagcgtatacacttcattttaggttttgaatttgatttgatttttgctgagtttgtttttgaaccctaatttggtgatgttgtacctggacttgcagctgcaccttcagcgactccatcggtatggtaataattgtgatttttgtcttctcgtgctaattcaaagttcttgaggttcattttttgctttttgtttgggataaatcagacgccttcaagaaaaagtccaaggtctgcaccggcaagtaagtgtatatagaagtaaattaatgtgcatttctgtacattcatgctttggacatgtagttatgttgctgtacttttgtttcagtcaattttttggtgtcaatttgaaactagttgggttaactatatgaatccttctaaatacattcgtttttttcgagcccaatcctatatttctatatgatgggagaatatatgtgtttcagcttcagtgcagaagccaaaacaaaagaagcgttacaggccagggacagtggcacttcgagaaatcagacactttcagaagacgtgggatcttcttattccagctgctcctttcatcagacttgtaattttctttcatatggacttatagcatatcaaagttgtttttaatgcattttttttggttacattcatcatggttctgttgatgttgtgcttgcttacaggaatacagttggactgctagatatagttagagcttagcagccatatgattagatgaaataggagaaactcaaccaattatagcttgattgagtgcttcattaccatgtaatctaactgaaatggaagaatgaacttaatgtcgaatctaaatagttgcaggtgtctaatttaatgtagattatggaaaaaagtgagagggctatcagtgttatgccttagttgagtggagaatatgtgaatcactgctgcactagcggataaagctaatgttaaggctttccactagttgatgaatattagttatactaaattgaaaagaaaaaaaaagatgctgaaaggatagtaaaaatttaaaaatactatggaatggagagaatagcccccttttcttgaataaaaaccctttaaaagaaggtatctttggcaagcttcaattgatgtaatactccataactcagtttctcattttcctttggactgcctcaagatgaaaattcacctattttctaactgatttagtttctcaatggagagcacactgatgttaacaaatagaagaaagaaaaactcaattctctttcttgagtttatctttatactctagtattttatttgtttgggcttggctgagttcaatttgcaggttagtttgaccagtttttgggattatgcactattgtcaaagaaaggtttccgtttcgtattgattcaatatgggattggaatgggaagaccaattatgatctgccggactttcaaaagtggaaactagttaactcttttcaattagtgggatctgcttctgtaggaggtttgataatttcagattttaagaaggtgtactacacagatctgctagtcaaaaaaagtttctacctcctgctctatatttcaggaagtccctgggactcaatttgtgattctggcagaaagtgtggactaagactgtataccaaaatctgcattctgattattctttaaatgtaaatgtgttgtagaactgctatattatacccttgatgataatatggtttagtttagaccaaataggttatggatttatccttgccttaattaaattttaaccaaacctaattgttttatccctcttttctgactggggcaggttagagaaattagtcacttttatgcacctggggtaactcgttggcaagctgaggcgttaattgctattcaagaggtatgccaatttctcaccataaaaagtggatctgttagctgctatttggatcgtgtgcattaattttaagttatttataggaatcatgtgttcctagagaggagagggaaatcgaatcatataaatatatttgctctcatatatcatcttgtagttttatatgtcaataacatatctagcttgaaatagtctctgctccatagacatattaaaattattttaatgtatcttagagtaattctactatcaatttcaaaatgtgcttaataatctcctataaaaggtttctccaagttatagaaaattacagcatgaactccataataatataaagtgtgttccaattgtgtggtcagggtagtaacggtgtgcgaaaagagccaacatattattaaagtgatcttcctcctttcggtacgtagcatacattaggtaagaaacgctatgttactcggactcttcaaaaatgatcccacacccatgccaaattcttcaacaatgcattagtttctgagaattcaacatgcacctgttgacatttttgaagagtccaaccaacatagaagaaagacaattgatatcatctcctaacaggcaatccaagatgggtcaggcaattagttagctaagggagtttgagtttctggaagatttatcatatttagagatgtctatcccaaactcttgctccatgaaggtttttctactttgattatggttgtgcatttttctcatctgagtcctgctcaactgaattgctttatatcttcaaattgcaaaaatactatttaggtgtattaatctggcaagttcaaagtaaaaactacttatctttgatttttgctttagataagcttctgtcctgaatggtacagtccagtgtccacaaataaagttgctactcggcttacccctaaatcagggtgtcaactccttttggatattgttcctatcgaggggacatatattatttctaagtaccactaattcctattggtttttgttttgatgtcataaatacaatacatatacttgcatcaacatacgagtatgactgatcttgcagcaacaagttattcaccttttcctctgaagctaggccaattctgatacatagtctctagcatggcataccttatttttttaagttccccggcaaagaataaggcattgaaaagagaagggatttcttagtgggtatttgctatacttgcatcaacatggtagaataccctatggcctccctaatatgcccattatttattaaagcactttgtgttttaaacctttttgactgcatgtataataagtgtatttgctatgtttatgaaaagatgtagttctttaatattttgtcagtgacaagttgatgtccagtaaacatgtggagtaaaaggaattggcttgttgtgatcattgatcagaaacagatagaatcccattaaactggtagggcgctgtggataactagtgagagaaaaggatagagctaagcatcagttttagcatgtctctctgcaagcatgcatttctgctccctataaaattttgtttgtatctggaagagcatcaatgatcttgtgaaataatgtactttgtgtcttctcaggaacctaggaaagtttctcaaatcctttgttctgtcagtagctatgctcaaataacagacatttagtcataggggaacaatcagcagtagaaacttgaaatgtctatttgtattcaaaagaaattatatttagtgttgatacatgaactattattcatcttcattctgtgcaatctgatgcatagtttggagtgaaagaactctactccagtcttcatacagattcctcaagagtcaaaacagtgttgcaatcatgtcaaactctgcttattctgtcagggcaatttgtctttacctgcacatatagtatattaataatcctgtgatccttgcacatcatacttttcttgggatgttttcatcattaatatcattaaaatagcaaaatgtagatggatgcataaagtaagaaagtttgtgtgcatagtctagttcagtcaacattattattgtcagcctacttttctagaaatgcctttttttataaaacatatatgtcatatagaactctattcttaatcaatatcagcaagagtgatataggtattttgcaggctgctgaagattttttagttcatttgtttgaagatgcaatgctatgtgctattcatgcgaagcgtgttacacttagtaagtttcctcttcaaattcttcctttttttggttttcttattgttccttctccacagttgacctaattctacatccctctactcattttcttgttgctttgaggaatgggctggcagtctggttcatcaaatcacttgtctttcacaaattatgttattctgttgtagatttcatatacctattagctttctcaattgtgttatgcttgtcctcctcagggaagaggaggaaatgctctacaaatgcttttagtcgcgaagttacggcatacaaattatgccatagatgcacatatattcgggatcctagttttatttactccatatatacatttgctaaagatgcaaaagtgaaatgaggcaatcttttctattgcctcttccaagttaacttctttcatatgaattcttcaatcacatctaagaggagtacctaagggccaggtacagcctgtttcgatatgggagattttaggtgtttagtgcagtgaaagttagcccaatgatcttttaagataacactgggaaacagaggagagcagttgccttagggtctcaactacacttatatggataatgaacacctgatgttctgaacaatacttctattattagtggtctggaatatcttgggcatctctaggtcttttagtactgcctacttgtttgcttggcatgctttgttatatgatgcgtaatttatgttgtttcattattcttgatgcacagtgaaaaaagattttgagctggctcgacgacttggaggaaaaggacaaccttggtga

SEQ ID NO: 10 - DNA, 5193 bp, Solanum lycopersicum
atggcgagaaccaaacacctcgcggaacgaagtcgcaccacttctggtacttctctacctctcctttttataatttaacctaactgcacacatatcgtaattttagagttttgaaaaagattagcgaattcgcaaatacaagtaagaagatctgacttagtttttaatacatatttgtaaagaaaaaaaatcaactgtatatggctcaagttgaagataagaagcggtgattaatacctttaatgtaaattcagcgatggtttctctgtgtgtgtgtgaatgtttacattagcgtatacacttcattttaggttttgaatttgatttgatttttgctgagtttgtttttgaaccctaatttggtgatgttgtacctggacttgcagctgcaccttcagcgactccatcggtatggtaataattgtgatttttgtcttctcgtgctaattcaaagttcttgaggttcattttttgctttttgtttgggataaatcagacgccttcaagaaaaagtccaaggtctgcaccggcaagtaagtgtatatagaagtaaattaatgtgcatttctgtacattcatgctttggacatgtagttatgttgctgtacttttgtttcagtcaattttttggtgtcaatttgaaactagttgggttaactatatgaatccttctaaatacattcgtttttttcgagcccaatcctatatttctatatgatgggagaatatatgtgtttcagcttcagtgcagaagccaaaacaaaagaagcgttacaggccagggacagtggcacttcgagaaatcagacactttcagaagacgtgggatcttcttattccagctgctcctttcatcagacttgtaattttctttcatatggacttatagcatatcaaagttgtttttaatgcattttttttggttacattcatcatggttctgttgatgttgtgcttgcttacaggaatacagttggactgctagatatagttagagcttagcagccatatgattagatgaaataggagaaactcaaccaattatagcttgattgagtgcttcattaccatgtaatctaactgaaatggaagaatgaacttaatgtcgaatctaaatagttgcaggtgtctaatttaatgtagattatggaaaaaagtgagagggctatcagtgttatgccttagttgagtggagaatatgtgaatcactgctgcactagcggataaagctaatgttaaggctttccactagttgatgaatattagttatactaaattgaaaagaaaaaaaaagatgctgaaaggatagtaaaaatttaaaaatactatggaatggagagaatagcccccttttcttgaataaaaaccctttaaaagaaggtatctttggcaagcttcaattgatgtaatactccataactcagtttctcattttcctttggactgcctcaagatgaaaattcacctattttctaactgatttagtttctcaatggagagcacactgatgttaacaaatagaagaaagaaaaactcaattctctttcttgagtttatctttatactctagtattttatttgtttgggcttggctgagttcaatttgcaggttagtttgaccagtttttgggattatgcactattgtcaaagaaaggtttccgtttcgtattgattcaatatgggattggaatgggaagaccaattatgatctgccggactttcaaaagtggaaactagttaactcttttcaattagtgggatctgcttctgtaggaggtttgataatttcagattttaagaaggtgtactacacagatctgctagtcaaaaaaagtttctacctcctgctctatatttcaggaagtccctgggactcaatttgtgattctggcagaaagtgtggactaagactgtataccaaaatctgcattctgattattctttaaatgtaaatgtgttgtagaactgctatattatacccttgatgataatatggtttagtttagaccaaataggttatggatttatccttgccttaattaaattttaaccaaacctaattgttttatccctcttttctgactggggcaggttagagaaattagtcacttttatgcacctggggtaactcgttggcaagctgaggcgttaattgctattcaagaggtatgccaatttctcaccataaaaagtggatctgttagctgctatttggatcgtgtgcattaattttaagttatttataggaatcatgtgttcctagagaggagagggaaatcgaatcatataaatatatttgctctcatatatcatcttgtagttttatatgtcaataacatatctagcttgaaatagtctctgctccatagacatattaaaattattttaatgtatcttagagtaattctactatcaatttcaaaatgtgcttaataatctcctataaaaggtttctccaagttatagaaaattacagcatgaactccataataatataaagtgtgttccaattgtgtggtcagggtagtaacggtgtgcgaaaagagccaacatattattaaagtgatcttcctcctttcggtacgtagcatacattaggtaagaaacgctatgttactcggactcttcaaaaatgatcccacacccatgccaaattcttcaacaatgcattagtttctgagaattcaacatgcacctgttgacatttttgaagagtccaaccaacatagaagaaagacaattgatatcatctcctaacaggcaatccaagatgggtcaggcaattagttagctaagggagtttgagtttctggaagatttatcatatttagagatgtctatcccaaactcttgctccatgaaggtttttctactttgattatggttgtgcatttttctcatctgagtcctgctcaactgaattgctttatatcttcaaattgcaaaaatactatttaggtgtattaatctggcaagttcaaagtaaaaactacttatctttgatttttgctttagataagcttctgtcctgaatggtacagtccagtgtccacaaataaagttgctactcggcttacccctaaatcagggtgtcaactccttttggatattgttcctatcgaggggacatatattatttctaagtaccactaattcctattggtttttgttttgatgtcataaatacaatacatatacttgcatcaacatacgagtatgactgatcttgcagcaacaagttattcaccttttcctctgaagctaggccaattctgatacatagtctctagcatggcataccttatttttttaagttccccggcaaagaataaggcattgaaaagagaagggatttcttagtgggtatttgctatacttgcatcaacatggtagaataccctatggcctccctaatatgcccattatttattaaagcactttgtgttttaaacctttttgactgcatgtataataagtgtatttgctatgtttatgaaaagatgtagttctttaatattttgtcagtgacaagttgatgtccagtaaacatgtggagtaaaaggaattggcttgttgtgatcattgatcagaaacagatagaatcccattaaactggtagggcgctgtggataactagtgagagaaaaggatagagctaagcatcagttttagcatgtctctctgcaagcatgcatttctgctccctataaaattttgtttgtatctggaagagcatcaatgatcttgtgaaataatgtactttgtgtcttctcaggaacctaggaaagtttctcaaatcctttgttctgtcagtagctatgctcaaataacagacatttagtcataggggaacaatcagcagtagaaacttgaaatgtctatttgtattcaaaagaaattatatttagtgttgatacatgaactattattcatcttcattctgtgcaatctgatgcatagtttggagtgaaagaactctactccagtcttcatacagattcctcaagagtcaaaacagtgttgcaatcatgtcaaactctgcttattctgtcagggcaatttgtctttacctgcacatatagtatattaataatcctgtgatccttgcacatcatacttttcttgggatgttttcatcattaatatcattaaaatagcaaaatgtagatggatgcataaagtaagaaagtttgtgtgcatagtctagttcagtcaacattattattgtcagcctacttttctagaaatgcctttttttataaaacatatatgtcatatagaactctattcttaatcaatatcagcaagagtgatataggtattttgcaggctgctgaagattttttagttcatttgtttgaagatgcaatgctatgtgctattcatgcgaagcgtgttacacttagtaagtttcctcttcaaattcttcctttttttggttttcttattgttccttctccacagttgacctaattctacatccctctactcattttcttgttgctttgaggaatgggctggcagtctggttcatcaaatcacttgtctttcacaaattatgttattctgttgtagatttcatatacctattagctttctcaattgtgttatgcttgtcctcctcagggaagaggaggaaatgctctacaaatgcttttagtcgcgaagttacggcatacaaattatgccatagatgcacatatattcgggatcctagttttatttactccatatatacatttgctaaagatgcaaaagtgaaatgaggcaatcttttctattgcctcttccaagttaacttctttcatatgaattcttcaatcacatctaagaggagtacctaagggccaggtacagcctgtttcgatatgggagattttaggtgtttagtgcagtgaaagttagcccaatgatcttttaagataacactgggaaacagaggagagcagttgccttagggtctcaactacacttatatggataatgaacacctgatgttctgaacaatacttctattattagtggtctggaatatcttgggcatctctaggtcttttagtactgcctacttgtttgcttggcatgctttgttatatgatgcgtaatttatgttgtttcattattcttgatgcacagtgaaaaaagattttgagctggctcgacgacttggaggaaaaggacaaccttggtga

SEQ ID NO: 11 - protein, 146 aa, Artificial Sequence
  Description of Artificial Sequence Synthetic Monocotyledon CenH3 consensus sequence
  MOD_RES (19)..(19): Any naturally occurring amino acid
  MOD_RES (46)..(46): Any naturally occurring amino acid
  MOD_RES (93)..(93): Any naturally occurring amino acid
  MOD_RES (112)..(112): Any naturally occurring amino acid
  MOD_RES (130)..(130): Any naturally occurring amino acid
MARTKHPAVRKPKKKLQFXRAPSTPGGASTSATPATGDRAAGTGVXKKHRFRPGTVALREIRKYQKSTELLIPFAPFVRLVREITNFYSKEVXRWTPEALLALQEAAEFHLXNLFEVANLCAIHAKRVTXMQKDIQLARRIGGRRW

SEQ ID NO: 12 - protein, 164 aa, Oryza sativa
MARTKHPAVRKSKAEPKKKLQFERSPRPSKAQRAGGGTGTSATTRSAAGTSASGTPRQQTKQRKPHRFRPGTVALREIRKFQKTTELLIPFAPFSRLVREITDFYSKDVSRWTLEALLALQEAAEYHLVDIFEVSNLCAIHAKRVTIMQKDMQLARRIGGRRPW

SEQ ID NO: 13 - protein, 164 aa, Oryza sativa
MARTKHPAMRKSKAEPKKKLQFERSPRPSKAQRAGGGTGTSATTRSAAGTSASGTPRQQTKQRKPHRFRPGTVALREIRKFQKTTELLIPFAPFSRLVREITDFYSKDVSRWTLEALLALQEAAEYHLVDIFEVSNLCAIHAKRVTIMQKDMQLARRIGGRRPW

SEQ ID NO: 14 - protein, 164 aa, Oryza sativa
MARTKHPAVRKSKAESKKKLQFERSPRPSKAQRAGGGTGTSATTRSAAGTSASGTPRQQTKQRKPHRFRPGTVALREIRKFQKTTELLIPFAPFSRLVREITDFYSKDVSRWTLEALLALQEAAEYHLVDIFEVSNLCAIHAKRVTIMQKDMQLARRIGGRRPW

SEQ ID NO: 15 - protein, 164 aa, Oryza sativa
MARTKHPAVRKSKAEPKKKLQFERSLRPSKAQRAGGGTGTSATTRSAAGTSASGTPRQQTKQRKPHRFRPGTVALREIRKFQKTTELLIPFAPFSRLVREITDFYSKDVSRWTLEALLALQEAAEYHLVDIFEVSNLCAIHAKRVTIMQKDMQLARRIGGRRPW

SEQ ID NO: 16 - DNA, 495 bp, Oryza sativa
atggctcgcacgaagcacccggcggtgaggaagtcgaaggcggagcccaagaagaagctccagttcgaacgctcccctcggccgtcgaaggcgcagcgcgctggtggcggcacgggtacctcggcgaccacgaggagcgcggctggaacatcggcttcagggacgcctaggcagcaaacgaagcagaggaagccacaccgcttccgtccaggcacagtggcactgcgggagatcaggaaatttcagaaaaccaccgaactgctgatcccgtttgcaccattttctcggctggtcagggagatcactgatttctattcaaaggatgtgtcacggtggacccttgaagctctccttgcattgcaagaggcagcagaataccacttagtggacatatttgaagtgtcaaatctctgcgccatccatgctaagcgtgttaccatcatgcaaaaggacatgcaacttgccaggcgtatcggtgggcggaggccatggtga

SEQ ID NO: 17 - DNA, 495 bp, Oryza sativa
atggctcgcacgaagcacccggcgatgaggaagtcgaaggcggagcccaagaagaagctccagttcgaacgctcccctcggccgtcgaaggcgcagcgcgctggtggcggcacgggtacctcggcgaccacgaggagcgcggctggaacatcggcttcagggacgcctaggcagcaaacgaagcagaggaagccacaccgcttccgtccaggcacagtggcactgcgggagatcaggaaatttcagaaaaccaccgaactgctgatcccgtttgcaccattttctcggctggtcagggagatcactgatttctattcaaaggatgtgtcacggtggacccttgaagctctccttgcattgcaagaggcagcagaataccacttagtggacatatttgaagtgtcaaatctctgcgccatccatgctaagcgtgttaccatcatgcaaaaggacatgcaacttgccaggcgtatcggtgggcggaggccatggtga

SEQ ID NO: 18 - DNA, 495 bp, Oryza sativa
atggctcgcacgaagcacccggcggtgaggaagtcgaaggcggagtccaagaagaagctccagttcgaacgctcccctcggccgtcgaaggcgcagcgcgctggtggcggcacgggtacctcggcgaccacgaggagcgcggctggaacatcggcttcagggacgcctaggcagcaaacgaagcagaggaagccacaccgcttccgtccaggcacagtggcactgcgggagatcaggaaatttcagaaaaccaccgaactgctgatcccgtttgcaccattttctcggctggtcagggagatcactgatttctattcaaaggatgtgtcacggtggacccttgaagctctccttgcattgcaagaggcagcagaataccacttagtggacatatttgaagtgtcaaatctctgcgccatccatgctaagcgtgttaccatcatgcaaaaggacatgcaacttgccaggcgtatcggtgggcggaggccatggtga

SEQ ID NO: 19 - DNA, 495 bp, Oryza sativa
atggctcgcacgaagcacccggcggtgaggaagtcgaaggcggagcccaagaagaagctccagttcgaacgctcccttcggccgtcgaaggcgcagcgcgctggtggcggcacgggtacctcggcgaccacgaggagcgcggctggaacatcggcttcagggacgcctaggcagcaaacgaagcagaggaagccacaccgcttccgtccaggcacagtggcactgcgggagatcaggaaatttcagaaaaccaccgaactgctgatcccgtttgcaccattttctcggctggtcagggagatcactgatttctattcaaaggatgtgtcacggtggacccttgaagctctccttgcattgcaagaggcagcagaataccacttagtggacatatttgaagtgtcaaatctctgcgccatccatgctaagcgtgttaccatcatgcaaaaggacatgcaacttgccaggcgtatcggtgggcggaggccatggtga

SEQ ID NO: 20 - DNA, 2166 bp, Oryza sativa
atggctcgcacgaagcacccggcggtgaggaagtcgaaggcggagcccaagaagaagctccagttcgaacgctcccctcggccgtcgaaggcgcagcgcgctggtggtgagcgcgcgctctctccccctctgcgtttctttttttttttcctttttctttcaatggcggtggatggtgaagcttatgccccccccccccccttcccgcctcttgcttgtcccctttgcaggcggcacgggtacctcggcgaccacggtgcgtgcgggagcgggtctttcgtttggtgattttttgattttgtggggggatatgtttttgttttgtatcttggctggatggatggcttgctcaccacctgtttgatggaatgcagaggagcgcggctggaacatcggcttcaggtgcgttctcttgggggggtttctagggttattcatgggctcgttggagcttttcctttctgtctcttggattccgggggacctgaggggctcaatgtgtcccttttcttgctctgttttaccgtgtgctgtactttcctcatcgttgttttctgaatatattataagaacagtagttgcagaaagatcttcaattgctcatcagtcaaagcttttcttgttttcattctgaaataatagcaaatccagtttggtccatggaggggttatctgaaacattatgaccataaaacatggtattaagcattgctagccaagaaatgtgtggtttttagacacgatgttgataggtgatttttatgctcatccattattagtctttgcatcgtgggaactgatttagtaaactttctttagtgtcatggttcaaatagcgtctgttctacctagatgataggtatccatatggaagtcttggctttggaattgctctccttttgttctcctgtgattaaataactttaacatgtgtgtgaagcagggacgcctaggcagcaaacgaagcagaggaagccacaccgcttccgtccaggcacagtggcactgcgggagatcaggaaatttcagaaaaccaccgaactgctgatcccgtttgcaccattttctcggctggtgggtacatcctgaacctgccttctctctatatcaaatatttcgtagtgcaaacttgtgtgatggaagctttttgtgccgataaaatttgcaggtcagggagatcactgatttctattcaaaggatgtgtcacggtggacccttgaagctctccttgcattgcaagaggtcagtggtcaaacctgtttattataagtttacaactgatggcttagttagggaagggtcagactgaattatactgtttaaattccattctgcttcaagactcaagtcacggctcaagagtgtaactgaaaaatgtacaaatcttccatgatcaataaaatgaatatctctgtgtgttgatttatgagtcagattgctaaattattatcctttttcagtagaacacctatatactacaaatatgcaacctccctattttgttgtgtctgttcaagattgctatcatagagtataccaatttcagttccttctttccagccatgtctgtttctgcataaccaggaaaaggaacaaagagctgacttaattctcacaaaataaattatgttatttacttgctgtcctgcaaatttccagtggttttccctctcctgcaggcagcagaataccacttagtggacatatttgaagtgtcaaatctctgcgccatccatgctaagcgtgttaccatcagtaagttgtcattctgaatgaacttttctctttctttttctccctttatattattatgctaaatggatatcatatatgccacagcctacatgatatcatatacgcatccacttcaaaagcattctatttttttataggaataacattctaattgcaggatgattcttaatacatgtgtttatatttaatgtcatatctagttttcatactcttaaatttatcatgattattgattaaacatagggagaattagttggtttgtgagttttgaggtgtgaaatatgctgcttgctattccctgtaaagcttatcagcgttgtcattgtgtggtttaacaaataaacgtttgttctgcagtgcaaaaggacatgcaacttgccaggcgtatcggtgggcggaggccatggtga

SEQ ID NO: 21 - DNA, 2166 bp, Oryza sativa
atggctcgcacgaagcacccggcgatgaggaagtcgaaggcggagcccaagaagaagctccagttcgaacgctcccctcggccgtcgaaggcgcagcgcgctggtggtgagcgcgcgctctctccccctctgcgtttctttttttttttcctttttctttcaatggcggtggatggtgaagcttatgccccccccccccccttcccgcctcttgcttgtcccctttgcaggcggcacgggtacctcggcgaccacggtgcgtgcgggagcgggtctttcgtttggtgattttttgattttgtggggggatatgtttttgttttgtatcttggctggatggatggcttgctcaccacctgtttgatggaatgcagaggagcgcggctggaacatcggcttcaggtgcgttctcttgggggggtttctagggttattcatgggctcgttggagcttttcctttctgtctcttggattccgggggacctgaggggctcaatgtgtcccttttcttgctctgttttaccgtgtgctgtactttcctcatcgttgttttctgaatatattataagaacagtagttgcagaaagatcttcaattgctcatcagtcaaagcttttcttgttttcattctgaaataatagcaaatccagtttggtccatggaggggttatctgaaacattatgaccataaaacatggtattaagcattgctagccaagaaatgtgtggtttttagacacgatgttgataggtgatttttatgctcatccattattagtctttgcatcgtgggaactgatttagtaaactttctttagtgtcatggttcaaatagcgtctgttctacctagatgataggtatccatatggaagtcttggctttggaattgctctccttttgttctcctgtgattaaataactttaacatgtgtgtgaagcagggacgcctaggcagcaaacgaagcagaggaagccacaccgcttccgtccaggcacagtggcactgcgggagatcaggaaatttcagaaaaccaccgaactgctgatcccgtttgcaccattttctcggctggtgggtacatcctgaacctgccttctctctatatcaaatatttcgtagtgcaaacttgtgtgatggaagctttttgtgccgataaaatttgcaggtcagggagatcactgatttctattcaaaggatgtgtcacggtggacccttgaagctctccttgcattgcaagaggtcagtggtcaaacctgtttattataagtttacaactgatggcttagttagggaagggtcagactgaattatactgtttaaattccattctgcttcaagactcaagtcacggctcaagagtgtaactgaaaaatgtacaaatcttccatgatcaataaaatgaatatctctgtgtgttgatttatgagtcagattgctaaattattatcctttttcagtagaacacctatatactacaaatatgcaacctccctattttgttgtgtctgttcaagattgctatcatagagtataccaatttcagttccttctttccagccatgtctgtttctgcataaccaggaaaaggaacaaagagctgacttaattctcacaaaataaattatgttatttacttgctgtcctgcaaatttccagtggttttccctctcctgcaggcagcagaataccacttagtggacatatttgaagtgtcaaatctctgcgccatccatgctaagcgtgttaccatcagtaagttgtcattctgaatgaacttttctctttctttttctccctttatattattatgctaaatggatatcatatatgccacagcctacatgatatcatatacgcatccacttcaaaagcattctatttttttataggaataacattctaattgcaggatgattcttaatacatgtgtttatatttaatgtcatatctagttttcatactcttaaatttatcatgattattgattaaacatagggagaattagttggtttgtgagttttgaggtgtgaaatatgctgcttgctattccctgtaaagcttatcagcgttgtcattgtgtggtttaacaaataaacgtttgttctgcagtgcaaaaggacatgcaacttgccaggcgtatcggtgggcggaggccatggtga

SEQ ID NO: 22 - DNA, 2166 bp, Oryza sativa
atggctcgcacgaagcacccggcggtgaggaagtcgaaggcggagtccaagaagaagctccagttcgaacgctcccctcggccgtcgaaggcgcagcgcgctggtggtgagcgcgcgctctctccccctctgcgtttctttttttttttcctttttctttcaatggcggtggatggtgaagcttatgccccccccccccccttcccgcctcttgcttgtcccctttgcaggcggcacgggtacctcggcgaccacggtgcgtgcgggagcgggtctttcgtttggtgattttttgattttgtggggggatatgtttttgttttgtatcttggctggatggatggcttgctcaccacctgtttgatggaatgcagaggagcgcggctggaacatcggcttcaggtgcgttctcttgggggggtttctagggttattcatgggctcgttggagcttttcctttctgtctcttggattccgggggacctgaggggctcaatgtgtcccttttcttgctctgttttaccgtgtgctgtactttcctcatcgttgttttctgaatatattataagaacagtagttgcagaaagatcttcaattgctcatcagtcaaagcttttcttgttttcattctgaaataatagcaaatccagtttggtccatggaggggttatctgaaacattatgaccataaaacatggtattaagcattgctagccaagaaatgtgtggtttttagacacgatgttgataggtgatttttatgctcatccattattagtctttgcatcgtgggaactgatttagtaaactttctttagtgtcatggttcaaatagcgtctgttctacctagatgataggtatccatatggaagtcttggctttggaattgctctccttttgttctcctgtgattaaataactttaacatgtgtgtgaagcagggacgcctaggcagcaaacgaagcagaggaagccacaccgcttccgtccaggcacagtggcactgcgggagatcaggaaatttcagaaaaccaccgaactgctgatcccgtttgcaccattttctcggctggtgggtacatcctgaacctgccttctctctatatcaaatatttcgtagtgcaaacttgtgtgatggaagctttttgtgccgataaaatttgcaggtcagggagatcactgatttctattcaaaggatgtgtcacggtggacccttgaagctctccttgcattgcaagaggtcagtggtcaaacctgtttattataagtttacaactgatggcttagttagggaagggtcagactgaattatactgtttaaattccattctgcttcaagactcaagtcacggctcaagagtgtaactgaaaaatgtacaaatcttccatgatcaataaaatgaatatctctgtgtgttgatttatgagtcagattgctaaattattatcctttttcagtagaacacctatatactacaaatatgcaacctccctattttgttgtgtctgttcaagattgctatcatagagtataccaatttcagttccttctttccagccatgtctgtttctgcataaccaggaaaaggaacaaagagctgacttaattctcacaaaataaattatgttatttacttgctgtcctgcaaatttccagtggttttccctctcctgcaggcagcagaataccacttagtggacatatttgaagtgtcaaatctctgcgccatccatgctaagcgtgttaccatcagtaagttgtcattctgaatgaacttttctctttctttttctccctttatattattatgctaaatggatatcatatatgccacagcctacatgatatcatatacgcatccacttcaaaagcattctatttttttataggaataacattctaattgcaggatgattcttaatacatgtgtttatatttaatgtcatatctagttttcatactcttaaatttatcatgattattgattaaacatagggagaattagttggtttgtgagttttgaggtgtgaaatatgctgcttgctattccctgtaaagcttatcagcgttgtcattgtgtggtttaacaaataaacgtttgttctgcagtgcaaaaggacatgcaacttgccaggcgtatcggtgggcggaggccatggtga

SEQ ID NO: 23 - DNA, 2166 bp, Oryza sativa
atggctcgcacgaagcacccggcggtgaggaagtcgaaggcggagcccaagaagaagctccagttcgaacgctcccttcggccgtcgaaggcgcagcgcgctggtggtgagcgcgcgctctctccccctctgcgtttctttttttttttcctttttctttcaatggcggtggatggtgaagcttatgccccccccccccccttcccgcctcttgcttgtcccctttgcaggcggcacgggtacctcggcgaccacggtgcgtgcgggagcgggtctttcgtttggtgattttttgattttgtggggggatatgtttttgttttgtatcttggctggatggatggcttgctcaccacctgtttgatggaatgcagaggagcgcggctggaacatcggcttcaggtgcgttctcttgggggggtttctagggttattcatgggctcgttggagcttttcctttctgtctcttggattccgggggacctgaggggctcaatgtgtcccttttcttgctctgttttaccgtgtgctgtactttcctcatcgttgttttctgaatatattataagaacagtagttgcagaaagatcttcaattgctcatcagtcaaagcttttcttgttttcattctgaaataatagcaaatccagtttggtccatggaggggttatctgaaacattatgaccataaaacatggtattaagcattgctagccaagaaatgtgtggtttttagacacgatgttgataggtgatttttatgctcatccattattagtctttgcatcgtgggaactgatttagtaaactttctttagtgtcatggttcaaatagcgtctgttctacctagatgataggtatccatatggaagtcttggctttggaattgctctccttttgttctcctgtgattaaataactttaacatgtgtgtgaagcagggacgcctaggcagcaaacgaagcagaggaagccacaccgcttccgtccaggcacagtggcactgcgggagatcaggaaatttcagaaaaccaccgaactgctgatcccgtttgcaccattttctcggctggtgggtacatcctgaacctgccttctctctatatcaaatatttcgtagtgcaaacttgtgtgatggaagctttttgtgccgataaaatttgcaggtcagggagatcactgatttctattcaaaggatgtgtcacggtggacccttgaagctctccttgcattgcaagaggtcagtggtcaaacctgtttattataagtttacaactgatggcttagttagggaagggtcagactgaattatactgtttaaattccattctgcttcaagactcaagtcacggctcaagagtgtaactgaaaaatgtacaaatcttccatgatcaataaaatgaatatctctgtgtgttgatttatgagtcagattgctaaattattatcctttttcagtagaacacctatatactacaaatatgcaacctccctattttgttgtgtctgttcaagattgctatcatagagtataccaatttcagttccttctttccagccatgtctgtttctgcataaccaggaaaaggaacaaagagctgacttaattctcacaaaataaattatgttatttacttgctgtcctgcaaatttccagtggttttccctctcctgcaggcagcagaataccacttagtggacatatttgaagtgtcaaatctctgcgccatccatgctaagcgtgttaccatcagtaagttgtcattctgaatgaacttttctctttctttttctccctttatattattatgctaaatggatatcatatatgccacagcctacatgatatcatatacgcatccacttcaaaagcattctatttttttataggaataacattctaattgcaggatgattcttaatacatgtgtttatatttaatgtcatatctagttttcatactcttaaatttatcatgattattgattaaacatagggagaattagttggtttgtgagttttgaggtgtgaaatatgctgcttgctattccctgtaaagcttatcagcgttgtcattgtgtggtttaacaaataaacgtttgttctgcagtgcaaaaggacatgcaacttgccaggcgtatcggtgggcggaggccatggtga